(19) United States
(12) Patent Application Publication
(10) Pub. No.: US 2011/0217314 A1
(43) Pub. Date: Sep. 8, 2011

(54) Methods and Compositions for Modulating Apoptosis

(75) Inventor: Amy S. Lee, San Marino, CA (US)

(73) Assignee: UNIVERSITY OF SOUTHERN CALIFORNIA, Los Angeles, CA (US)

(21) Appl. No.: 12/896,119

(22) Filed: Oct. 1, 2010

Related U.S. Application Data
(63) Continuation of application No. 10/975,045, filed on Oct. 26, 2004, now abandoned.
(60) Provisional application No. 60/514,661, filed on Oct. 27, 2003.

(51) Int. Cl. A61K 39/395; C12N 5/071; C12N 5/09; A61P 35/00

(52) U.S. Cl. 424/172.1; 435/375

(57) ABSTRACT

This invention relates to compositions and methods for modulating apoptosis by regulating the activity of endoplasmic reticulum transmembrane glucose regulated protein 78 (GRP78).

### CROSS REFERENCE TO RELATED APPLICATIONS

This application is a continuation of U.S. Ser. No. 10/975,045, filed on Oct. 26, 2004, which claims benefit of priority under 35 U.S.C. §119 from U.S. Provisional Application Ser. No. 60/514,661, filed on Oct. 27, 2003, the disclosures of which are incorporated herein by reference.

### STATEMENT AS TO FEDERALLY-SPONSORED RESEARCH

This invention was made with government support under Grant Nos. CA 20607 and AI42394, awarded by the National Institutes of Health. The government has certain rights in the invention.

### TECHNICAL FIELD

This invention relates to methods and compositions for modulating apoptosis by selectively targeting glucose regulated proteins (GRPs) and more particularly to modulating the activity and/or interaction of GRP78 and procaspase.

### BACKGROUND

Resistance to chemotherapy remains a major obstacle for the treatment of cancer. The complexity of drug resistance in human cancer strongly suggests the involvement of multiple pathways. One mechanism, both intrinsic and acquired, is the result of genetic alterations within cancer cells. Another mechanism may result from environmental conditions that occur naturally in solid tumors. Because of poor vascularization, solid tumors usually contain regions undergoing glucose starvation and hypoxia, resulting in acidosis and alterations in cell metabolism. These pockets of hypoxia and nutrient deprivation occur in well differentiated, slow growing, non-metastatic tumors, as well as in rapidly growing, aggressive anaplastic malignancies.

Stress conditions in cell culture, such as glucose starvation, commonly cause the glucose-regulated stress response which, is part of a general cellular defense mechanism referred to as the unfolded protein response (UPR). One characteristic of the UPR is the induction of the endoplasmic reticulum (ER) resident stress proteins referred to as the glucose-regulated proteins (GRPs). The GRPs are Ca²⁺-binding chaperone proteins with protective properties. The best characterized GRP is GRP78, a 78-kDa protein also referred to as BiP. As a protein chaperone, GRP78 is known to form complexes with heterologous proteins that are processed through the ER.

Glucose Regulated Proteins, or GRPs (GRP74, GRP78, GRP94, GRP170, ERp72, PDI, calreticulin, and GRP58 (alias ERp57)) are ER molecular chaperones that assist in protein folding and assembly. GRP78, GRP94, ERp72 and calreticulin are also Ca2+ binding proteins. GRP78 and GRP94 share sequence homology with heat shock proteins. The GRP family of proteins is coordinately induced by glucose starvation, anoxia, alterations in intracellular calcium and, exposure to inhibitors of glycosylation as well as by PDT-mediated oxidative stress (Gomer, et al., Cancer Res. 51:6574-79, 1991; and Li, et al., J. Cell Physiol. 153:575-82, 1992). The 78,000 GRP (i.e., GRP78) is identical in sequence to the immunoglobulin heavy chain binding protein and both GRP78 and GRP94 are localized in the ER.

Many of the cytotoxic drugs, including topoisomerase inhibitors such as etoposide, initiate programmed cell death. DNA damaging agents such as etoposide can trigger cell death through the p53-mediated caspase cell death signaling cascade, resulting in cytochrome c release and the activation of caspase-3. Caspases-3, -6, and -7 are members of the apoptotic executing group of caspases with caspase-7 structurally and functionally most similar to caspase-3. Active caspase-7 has been shown to be associated with the mitochondria and the ER membranes, whereas caspase-3 remains cytosolic. Although these observations suggest that similar apoptotic executioner's function in different cellular compartments and act on distinct substrates, there is limited information on the contribution of organelles such as the ER in the apoptotic process.

Accordingly, there exists a need in the art to identify key interactions between proteins involved in the apoptotic pathway and to regulate those interactions.

### SUMMARY

Overexpression and antisense approaches in cell systems show that GRP78 can protect cells against cell death caused by disturbance of ER homeostasis. Whereas GRP78 overexpression could limit damage in normal tissues and organs exposed to ER stress, the anti-apoptotic function of GRP78 also predicts that its natural induction in neoplastic cells could lead to cancer progression and drug resistance. In a variety of cancer cell lines, solid tumors, and human cancer biopsies, the level of GRP78 is elevated, correlating with malignancy. Using human cancer and other cell lines, a large number of stress induction studies show that a glucose-regulated stress response results in the induction of GRP78 and other coordinately regulated GRP genes correlating with cellular drug resistance. Nonetheless, the direct role of GRPs in conferring drug resistance has not been proven. This is because of the inherent problems associated with using stress inducers or deficiencies in certain cell functions to induce the GRPs, because the inducing conditions can exert other unknown pleiotropic effects, possibly affecting multiple cellular pathways. Furthermore, the mechanisms for the protective function of the ER localized GRPs in drug resistance are not understood.

Methods and compositions for modulating apoptosis by regulating the physical and functional interactions of glucose responsive protein 78 (GRP78) with cytosolic components of a cell that mediate apoptosis are provided. The methods and compositions are particularly well suited to identifying agents that can be used in conjunction with apoptosis-inducing therapeutic compounds to treat cell proliferative disorders. Thus, the disclosure relates to the preparation of pharmaceutical compositions for treating, preventing, and/or delaying a disease in a subject, such as, for example, a cell proliferative disorder.

Accordingly, in one embodiment, the invention provides a method of modulating apoptosis by contacting glucose regulated protein 78 (GRP78) endoplasmic reticulum transmembrane protein with an agent that regulates the interaction of the transmembrane protein with a cytosolic component that mediates apoptosis. In one aspect, the cytosolic component is a caspase. The caspase can be, for example, caspase-7. In general, the method can be used to promote or inhibit apoptosis. The agent can be, for example, a polypeptide, an antibody or a small molecule. In one aspect, the agent interacts with the ATP-binding domain of GRP78.

In another embodiment, the invention provides a method of modulating apoptosis by contacting glucose regulated protein 78 (GRP78) with an agent that inhibits or prevents the ability of the protein to integrate in to the membrane of the endoplasmic reticulum. In one aspect, the agent interacts with a hydrophobic transmembrane domain III (amino acids 210-260 of SEQ ID NO:2) or domain IV (amino acids 400-450 of SEQ ID NO:2) of GRP78.

In a further embodiment, the invention provides a method of identifying an agent that modulates the interaction of glucose regulated protein 78 (GRP78) with a cytosolic component that mediates apoptosis. The method includes providing glucose regulated protein 78 (GRP78) integrally-associated with a membrane, providing a cytosolic component comprising at least one caspase, providing an agent, contacting the protein with the component and the agent simultaneously or in succession, and determining the effect of the agent on the interaction of the protein and the component as compared to a control.

In a further embodiment, the invention provides a method of identifying an agent that modulates the interaction of glucose regulated protein 78 (GRP78) with a membrane. The method includes providing a polypeptide comprising the hydrophobic transmembrane domain III (amino acids 210-260 of SEQ ID NO:2) and/or domain IV (amino acids 400-450 of SEQ ID NO:2) of the protein of glucose regulated protein 78 (GRP78), providing an agent, contacting the polypeptide with the agent, and determining the effect of the agent on the ability of the polypeptide to incorporate in to the membrane, as compared to a control.

In yet another embodiment, the invention provides a method of inhibiting apoptosis in a target tissue by overexpressing GRP78 or GRP94 in said tissue. Exemplary tissues include neuronal tissue, vascular tissue and cardiac tissue.

The invention provides a method of modulating apoptosis in a cell, the method comprising contacting a glucose regulated protein (GRP) with an agent that regulates the interaction of the GRP with a cytosolic component that mediates apoptosis.

The invention also provides a method of promoting apoptosis in a cell, the method comprising inhibiting glucose regulated protein 78 (GRP78) with an agent that (i) inhibits or prevents the ability of GRP78 to interact with a cytosolic protein and/or (ii) inhibits the production of GRP78.

The invention further provides a method of identifying an agent that modulates the interaction of glucose regulated protein 78 (GRP78) with a cytosolic component that mediates apoptosis. The method comprises (a) providing glucose regulated protein 78 (GRP78) integrally-associated with a membrane; (b) providing a cytosolic component comprising at least one caspase; (c) providing an agent; (d) contacting the protein of a) with the component of (b) and the agent of (c) simultaneously or in succession; and (e) determining the effect of the agent on the interaction of the protein and the component as compared to a control.

The invention provides a glucose regulated protein (GRP) inhibitory nucleic acid molecule comprising a nucleic acid that interacts with a glucose regulated protein (GRP) polynucleotide. In one aspect, the inhibitory nucleic acid is an antisense molecule. In another aspect, the nucleic acid is a small inhibitory nucleic acid (siNA) molecule.

The invention also provides a glucose regulated protein modulating agent comprising a soluble domain of a GRP protein.

The invention further provides a method of identifying an agent that modulates the interaction of glucose regulated protein 78 (GRP78) with a cytosolic component that mediates apoptosis, the method comprising: (a) providing a polypeptide comprising the ATP-binding domain of glucose regulated protein 78 (GRP78); (b) providing a cytosolic component comprising at least one caspase; (c) providing an agent; (d) contacting the polypeptide of a) with the component of (e) and the agent of (c) simultaneously or in succession; and (f) determining the effect of the agent on the interaction of the polypeptide and the component as compared to a control.

The invention yet further provides a method of identifying an agent that modulates the interaction of glucose regulated protein 78 (GRP78) with a membrane, the method comprising: (a) providing a polypeptide comprising the hydrophobic transmembrane domain III (amino acids 210-260 of SEQ ID NO:1 or 2) and/or domain IV (amino acids 400-450 of SEQ ID NO:1 or 2) of the protein of glucose regulated protein 78 (GRP78); (b) providing an agent; (c) contacting the polypeptide of a) the agent of b) simultaneously or in succession; and (d) determining the effect of the agent on the interaction of the polypeptide with the membrane as compared to a control.

The invention provides a method of modulating apoptosis, the method comprising contacting a cell comprising a caspase polypeptide with an agent that regulates the interaction of the polypeptide with glucose regulated protein 78 (GRP78) endoplasmic reticulum transmembrane protein.

The invention also provides a method of modulating apoptosis, the method comprising contacting a cell comprising a glucose regulated protein 94 (GRP94) endoplasmic reticulum transmembrane protein with an agent that regulates the interaction of the transmembrane protein with a cytosolic component that mediates apoptosis.

The invention further provides a method of inhibiting apoptosis in a target tissue, the method comprising overexpressing GRP78 or GRP94 in said tissue.

The invention provides a method of identifying an agent that modulates the interaction of glucose regulated protein 94 (GRP94) with a cytosolic component that mediates apoptosis, the method comprising: (a) providing glucose regulated protein 94 (GRP94); (b) providing a cytosolic component comprising at least one caspase; (c) providing an agent; (d) contacting the protein of (a) with the component of (b) and the agent of (c) simultaneously or in succession; and (e) determining the effect of the agent on the interaction of the protein and the component as compared to a control.

Also provided by the invention is a nucleic acid construct comprising a glucose regulated protein (GRP) inhibitory nucleic acid molecule operably linked to an expression control element.

In another aspect, the invention provides a recombinant vector comprising a nucleic acid construct comprising a glucose regulated protein (GRP) inhibitory nucleic acid molecule operably linked to an expression control element.

The invention provides a pharmaceutical composition comprising a nucleic acid construct of the invention in a pharmaceutically acceptable carrier.

A method for inhibiting cell proliferation is also provided by the invention. The method comprising contacting a target cell having a cell proliferative disorder with a nucleic acid construct of the invention.

In yet another aspect the invention provides a method for treating a cell proliferative disorder in a subject comprising administering to the subject a nucleic acid construct of the invention.

The invention provides a nucleic acid construct comprising a glucose regulated protein (GRP) polynucleotide operably linked to an expression control element.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1, Panel A depicts a comparison of GRP78 protein levels in parental CHO cell line and its derivative C.1. Total protein lysates (25 and 50 g/lane) were separated on 8% SDS-PAGE, and the levels of GRP78, GRP94, and protein X were determined by immunoblotting with an anti-KDEL antibody.

FIG. 1, Panel B depicts GRP78 localization by immunofluorescence. Subcellular distribution of GRP78 is primarily perinuclear, indicative of ER localization.

FIG. 1, Panel C depicts co-localization of GRP78 and caspase-7 in the ER.

FIG. 2, Panel A depicts clonogenic survival assays for CHO and C.1 cells subjected to various concentrations of etoposide for 6 h.

FIG. 2, Panel B depicts clonogenic survival assays for CHO and C.1 cells subjected to various concentrations of adriamycin for 1 h.

FIG. 2, Panel C depicts clonogenic survival assays for CHO and C.1 cells subjected to various concentrations of camptothecin for 24 h.

FIG. 3, Panel A depicts GRP78 overexpression protecting cells from etoposide-induced apoptosis. C.1 and CHO cells were either non-treated (Ctrl) or treated with 30 M etoposide (Etop) for 6 h. Viable cells are those with low annexin or no annexin and PI staining (lower left panel). Early stage apoptotic cells are represented by high annexin and low PI staining (lower right panel), later stage apoptotic cells represented by high annexin and high PI staining (upper right panel), and necrosis is represented by cells with high PI and low annexin staining (upper left panel).

FIG. 3, Panel B depicts DNA fragmentation pattern of CHO and C.1 cells following etoposide treatment.

FIG. 4, Panel A depicts overexpression of GRP78 confers etoposide resistance to human bladder carcinoma T24/83 cells. T24/83 cell lines stably transfected with the empty vector pcDNA (T24/83-pcDNA) or vector expressing wild-type GRP78 (T24/83-GRP78) were established. Immunoblot analysis of GRP78 protein level for the two cell lines is shown (inset).

FIG. 4, Panel B depicts immunofluorescence imaging of GRP78 expression in T24/83 cells. GRP78 localization is perinuclear.

FIG. 4, Panel C depicts the effect of overexpression of GRP78 on etoposide-induced apoptosis. T24/83-pcDNA and T24/83-GRP78 cells were either non-treated (Ctrl) or treated with etoposide (Etop).

FIG. 5, Panel A depicts the effect of GRP78 overexpression on topoisomerase II expression and caspase-7 activation. Total protein lysates were prepared from non-treated (Ctrl) or etoposide-treated (Etop) CHO and C.1 cells.

FIG. 5, Panel B depicts overexpression of GRP78 as inhibiting in vitro activation of caspase-7.

FIG. 5, Panel C depicts cytoplasmic extracts prepared from CHO and C.1 cells and incubated with (+) or without (−) 10 μM cytochrome c and the various amounts of dATP (mM) as indicated.

FIG. 6, Panel A depicts cell lysates from CHO and C.1 cells immunoprecipitated with anti-caspase-7 (lanes 1 and 2) or anti-caspase-3 (lanes 3 and 4) antibodies.

FIG. 6, Panel B depicts cell lysates in extraction buffer from CHO and AD-1 cells were immunoprecipitated with anti-caspase-7 antibody (lanes 1 and 2). Whole cell extracts (WCE) from CHO and AD-1 cells were immunoblotted in parallel (lanes 3 and 4). The positions of GRP78, procaspase-7, and the deletion mutant form of GRP78 (78) are indicated.

FIG. 6, Panel C depicts a schematic drawing of wild-type (WT) GRP78 and AD-1 showing the location of the signal sequence (S), the ATP-binding domain, and the AD-1 deletion spanning amino acids 175 to 201.

FIG. 7, Panel A depicts a hydropathicity plot of GRP78 as generated using the Kyte-Doolittle method with a window size of 17. Four putative hydrophobic domains (I-IV) are identified. Represented below is a schematic drawing of the mature GRP78 protein with the hydrophobic domains IV and III as putative transmembrane domains generating carboxyl 35- and 50-kDa trypsin resistant fragments.

FIG. 7, Panel B depicts limited trypsin digestion. Isolated microsomes from C.1 cells were either non-treated (lane 1) or subjected to trypsin digestion at the concentration of 0.01% (lane 2) or 0.05% (lane 2). At the end of the reaction, the amount of GRP78 was detected by Western blotting using the rabbit polyclonal anti-GRP78 antibody recognizing the carboxyl terminus (StressGen, Victoria, Canada) (left panel). The full-length GRP78 band is indicated by a closed arrow, and the 35- and 50-kDa proteolytic products are indicated by closed arrows highlighted with a star. The same membrane was stripped and re-probed with a rabbit polyclonal anti-calreticulin (CRT) antibody (middle panel) or a rabbit polyclonal anti-calnexin antibody recognizing the amino terminus of calnexin (right panel). The full-length CRT and calnexin are indicated by closed arrows, and the 70-kDa proteolytic product of calnexin is indicated by an open arrow highlighted by a star.

FIG. 7, Panel C depicts sodium carbonate extraction. The microsome (M) fraction was either non-treated (lane 1) or treated with 100 mM sodium carbonate and separated into pellet (P) and supernatant (S) fractions (lanes 2 and 3, respectively). The protein samples from each fraction were separated by 10% SDS-PAGE and subjected to Western blotting with rabbit anti-GRP78 antibody (left panel), rabbit anti-calreticulin antibody (middle panel), and rabbit anti-calnexin antibody (right panel).

FIG. 8, Panel A depicts cytoplasmic extracts (50 μg/lane) prepared from non-treated (Ctrl) and etoposide-treated (Etop) AD-1 and C.1 cells as separated on 10% SDS-PAGE and immunoblotted using anticaspase-7 antibody.

FIG. 8, Panel B depicts the DNA fragmentation pattern of C.1 and AD-1 cells following etoposide treatment.

FIG. 8, Panel C depicts cell death assays indicating that the ATP-binding domain of GRP78 is necessary for protection against etoposide-induced cell death. The S.D. is shown.

FIG. 9 depicts the relative cell cycle distribution of GRP-78. The percentage of cells in G1, G2, and S phase was determined.

FIG. 10 is a schematic showing the subcloning of the 320 bp grp78 exon 1 fragment.

FIG. 11A-C shows the schemes used for subcloning. (A) shows the scheme for the subcloning of full-length His-tagged GRP78 into pShuttle CMV adenovirus; (B) shows the scheme for the subcloning of full-length His-Tagged GRP78 Full-length antisense (AS); and (C) shows the scheme used for the subcloning of GRP78 (320 BP) antisense (AS) into PShuttle CMV adenovirus.

FIG. 12 shows the expression of His-tagged GRP78, GRP78(AS) and 320(AS) in adenovirus infected 293 T cells.

FIGS. 13A and B shows the results of transduced MDA-MB-435 cells treated with etoposide in the presence and absence of the 320(AS).

FIG. 14 shows a Western blot of 293T cells. The concentrations of siRNA Grp78 II used were indicated on top.

FIG. 15 shows a Western blot of MDA-MB-435 cells. The concentrations of siRNA Grp78 II used were indicated on top.

FIG. 16 shows suppression of GRP78 by siRNA oligonucleotides results in enhanced sensitivity to etoposide-mediated cell death in breast cancer cells.

FIG. 17A-B show the physical and functional interaction between GRP BlK. (A) 293T cells were either not treated (lanes 1, 2) or treated with 50 uM etoposide for 24 hours (lane 3). Cells were harvested and cells lysate immunoprecipitated with either goat IgG (lane 1) or goat anti-BlK antibody (lanes 2, 3). Western blots with anti-BlK antibody and anti-GRP78 antibody show co-immunoprecipitation of endogenous GRP78 with Blk. (B) 293T cells were transiently transfected with either pcDNA, CMV promoter driven expression vector for His-tagged GRP78, expression vector for Flag-tagged Glk-b5TM alone or in combination as indicated. After 48 hours, cell death was determined by trypan blue exclusion assay. The level of apoptosis observed in cells transfected with pcDNA3 was set as 1. The results showed overexpression of GRP78 protects 293T cells from death induced by transient transfection of ER-targeted Blk-b5μM.

### DETAILED DESCRIPTION

Within the microenvironment of a solid tumor, unique stress conditions can lead to induction of GRPs. The data and invention described herein indicate that GRPs act as anti-apoptotic proteins. The GRP-mediated protection involves GRP interaction with effectors of apoptosis, leading to the blockage of cell death induced by drug treatment.

Thus, in accordance with the invention, GRPs (e.g., GRP74, GRP78 and GRP94) represent rational targets for chemotherapeutics, immunotherapeutics, antisense, ribozymes, siRNA and vaccines relevant to the treatment of cell proliferative diseases such as cancer. In view of their function as molecular chaperones, the GRPs (e.g., GRP78 and 94) further represent rational targets for the development of therapeutics for tissue injury and stress, such as can occur in ischemic injuries including, but not limited to, organ (kidney, heart, lung, liver) transplantation, cerebral stroke, and myocardial infarct. Methods and compositions for modulating apoptosis are provided.

The invention provides methods and compositions useful to modulate apoptosis in a cell, tissue and/or subject. “Apoptosis” refers to programmed cell death which occurs by an active, physiological process. Apoptosis plays an important role in developmental processes, including morphogenesis, maturation of the immune system, and tissue homeostasis whereby cell numbers are limited in tissues that are continually renewed by cell division. Apoptosis is an important cellular safeguard against tumorigenesis. An apoptotic cell or a cell going through “programmed cell death” exhibits one or more characteristics associated with timed or targeted cell death. Characteristics include inhibition of cell survival, growth, death or differentiation, protein/nucleic acid cleavage/fragmentation, chromatin condensation, membrane fragmentation, changes in expression or activity of one or more proteins that promote apoptosis or that inhibit apoptosis.

“Modulating” apoptosis means increasing, stimulating or inducing, or decreasing, inhibiting, blocking or preventing (e.g., prophylaxis) one or more characteristics of programmed cell death as described herein or known in the art. For example, the methods and compositions of the disclosure include agents (e.g., antisense molecules, ribozymes, polypeptides, small molecules, and the like) that increase, stimulate or induce apoptosis by inhibiting the activity or production of GRPs. The disclosure also includes agents and methods that increase the activity or production of GRPs to inhibit apoptosis in tissues or cells subject to damage due to ischemia and the like.

GRP78 binds transiently to nascent, secretory and transmembrane proteins and binds permanently to abnormally folded or processed proteins in the ER. GRP78 is thought to have a protective function during and after cellular stress when protein processing in the ER is perturbed. GRP78 has been proposed as a possible target for several antitumor agents, principally radicicol and geldanamycin (Scheibel and Buckner, Biochem Pharm 56:675-82, 1998).

A potential yet heretofore uncharacterized protective role of GRP94 in ischemia is supported by the observation that expression of GRP94 is enhanced in hippocampus after transient forebrain ischemia of a duration known to result in neuronal death (Yagita et al., J Neurochem 72:1544-1551, 1999). GRP94 is similarly induced following acute kidney ischemia (Kuznetsov, Proc Natl Acad Sci USA 93:8584-8589, 1996). By comparison, heat-shock proteins, including HSP90, are over-expressed during the oxidative stress of reperfusion that generally follows ischemia. For example, the higher levels of GRP78 and GRP94 in the brains of immature rats when compared to those of adult animals account for the higher resistance of immature rats to seizure. In addition, specific induction of these GRPs in the dentate gyms region of the adult rat brain following seizure is associated with a neuroprotective effect. For early-onset familial Alzheimer's disease (FAD), overexpression of GRP78 in neuroblastoma cells expressing a mutant presenilin-1 (PS1) protein was reported to restore resistance to ER stress.

Expression of GRP78 also prevents the aggregation and facilitates the proteasomal degradation of mutant prion proteins, which are implicated in neurodegenerative disorders such as prion diseases and transmissible spongiform encephalopathies. Induction of GRP78 has also been observed in endothelial cells damaged by reductive stress that was caused by hyperhomocysteinaemia, which, with both genetic and environmental components, is a common risk factor for thrombotic vascular events such as premature arteriosclerosis, stroke, myocardial infarction, and thrombosis. Therefore, the induction of GRP could be an adaptive response evolved in mammals to protect endothelial cells against stress-induced cell death.

Although apoptosis is mediated by diverse signals and complex interactions of cellular gene products, the results of these interactions ultimately feed into a cell death pathway that is evolutionarily conserved between humans and invertebrates. The pathway, itself, is a cascade of proteolytic events analogous to that of the blood coagulation cascade.

Several gene families and products that are involved in the apoptotic pathway have been identified. Key to the apoptotic program is a family of cysteine proteases termed caspases. The human caspase family includes Ced-3, human ICE (interleukin-1beta converting enzyme) (caspase-1), ICH-1 (caspase-2), CPP32 (caspase-3), ICErelII (caspase-4), ICErelII (caspase-5), Mch2 (caspase-6), ICE-LAP3 (caspase-7), Mch5 (caspase-8), ICE-LAP6 (caspase-9), Mch4 (caspase-10), caspase 11-14 and others.

It has been demonstrated that caspases are required for apoptosis to occur. Moreover, caspases appear to be necessary for the accurate and limited proteolytic events that are the hallmark of classic apoptosis (see Salvesen and Dixit, Cell 91:443-446, 1997). During apoptosis, an initiator caspase zymogen is activated by autocatalytic cleavage, which then activates the effector caspases by cleaving their inactive zymogen (Salvesen and Dixit, Proc. Natl. Acad. Sci. USA 96:10964-10967, 1999; Srinivasula et al., Mol. Cell. 1:949-957, 1998). The effectors are responsible for proteolytic cleavage of a number of cellular proteins leading to the characteristic morphological changes and DNA fragmentation that are often associated with apoptosis (reviewed in Cohen, Biochem. J. 326:1-16, 1997; Henkart, Immunity 4:195-201, 1996; Martin and Green, Cell 82:349-352, 1995; Nicholson and Thomberry, TIBS 257:299-306, 1997; Porter et al., BioEssays 19:501-507, 1997; Salvesen and Dixit, Cell 91:443-446, 1997).

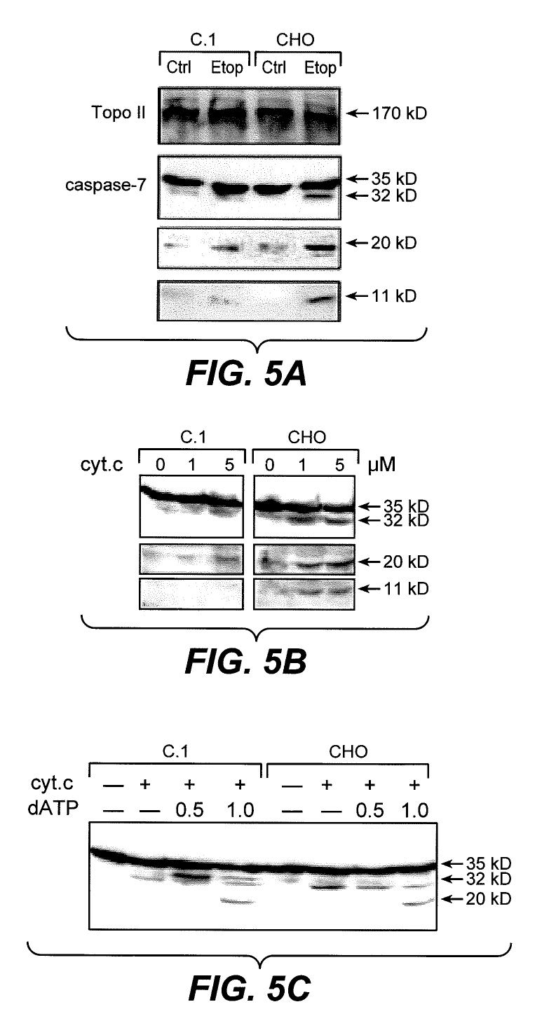

Among the executor caspases, caspase-7 has been reported to be associated with the ER. Upon induction of apoptosis, procaspase-7 (35 kDa) is first converted into a 32-kDa intermediate, which is further processed into active 20- and 11-kDa subunits. Western blotting data provided herein indicates that treating CHO cells with etoposide results in activation of caspase-7, giving rise to the 32-kDa intermediate form (FIG. 5, Panel A). Upon longer exposure of the autoradiogram, the active 20- and 11-kDa forms were evident in the etoposide-treated cells. When GRP78 was overexpressed, a low level of caspase-7 activation was detected in both the non-treated and etoposide-treated cells. These data indicate that GRP78 can suppress caspase-7 activation in vivo thereby inhibiting apoptosis. In addition, upon addition of cytochrome c, caspase-7 activation was higher in CHO cells than C.1 cells, as evidenced by the increase in the active 20- and 11-kDa forms in the CHO samples compared with the C.1 samples. In the presence of both cytochrome c and dATP, the suppressive effect of the C.1 samples was reversed. At 1 mM dATP, both cell lines showed equivalent amounts of the 32- and 20-kDa forms, indicating that dATP releases procaspase-7 from GRP78, resulting in its activation.

The invention demonstrates that over-expression in tissue culture systems of GRP78, GRP94 and adapt78 can protect cells against cell death. The invention also demonstrates that inhibitors of expression (e.g., antisense and RNAi) or inhibitors of GRP activity in tissue culture systems can induce apoptosis. Thus, the protective function of the GRPs is useful and beneficial in situations involving tissue or organ damage. This same protective function is detrimental in cancer by preventing apoptosis of cancer cells.

As demonstrated herein, GRP upregulation and/or overexpression is useful in limiting damage in organs exposed to stress. However, the anti-apoptotic function of GRPs also indicates that their induction in neoplastic cells and cell proliferative disorders could lead to cancer progression and drug resistance. In a variety of cancer cell lines, solid tumors and human cancer biopsies, the levels of GRP78 and GRP94 are elevated, correlating with malignancy. In addition, induction of GRP78 has been shown to protect cancer cells from immune surveillance, whereas suppressing the stress-mediated induction of GRP78 enhanced apoptosis, inhibited tumor growth and increased the cytotoxicity of chronic hypoxic cells.

Thus, the invention provides methods and compositions useful for targeted suppression of GRP expression or function in cancer cells as a novel approach to cancer therapy. For example, Genistein, which suppresses both the GRP and the heat shock responses, inhibits the growth of carcinogen-induced tumors in rats and in human leukemia cells transplanted into mice. In another example, GRP94 has been shown to associate with and stabilize p185/erbB2 (also referred to HER-2/neu), which is commonly over-expressed in breast carcinomas and is associated with poor prognosis. Treatment of breast cancer cells with geldanamycin, an anti-proliferative agent, enables the degradation of p185 in the breast cancer cells by disrupting the GRP94-p185 complex.

Pre-induction of GRP in a variety of human cancer cell lines confers resistance to inhibitors of topoisomerase II (e.g., etoposide) but increases sensitivity to DNA cross-linking agents such as cisplatin. Direct suppression of GRP94 levels by antisense knockdown strategies results in enhanced sensitivity to etoposide-induced cell death.

Accordingly, the invention provides methods and compositions useful in reducing the anti-apoptotic effect of GRPs, increase sensitivity of cancer cells to chemotherapeutic agents, and promote apoptosis of neoplastic cells. The methods and compositions of the invention inhibit the production or activity of GRPs in neoplastic cells (e.g., cancer cells) and tissues.

As will be discussed below, the invention provides the first evidence that a population of GRPs is integrally-associated with the membrane of the endoplasmic reticulum. These GRPs interact with a cytosolic component to mediate apoptosis. A “cytosolic component that mediates apoptosis”, as used herein, is any polypeptide, or group of polypeptides that cooperate in the initiation or facilitation of apoptosis. For example, the interaction between GRP78 and caspase-7 and/or the interaction between GRP94 and p185/erbB2 is involved in GRPs ability to modulate apoptosis. The interaction can be, for example, with caspase-7 individually, or as part of a group of other polypeptides involved in the apoptosis pathway.

The invention further provides the first evidence that complex formation between endogenous GRP78 and caspase-7 occurs in association with the endoplasmic reticulum. While the data provided herein indicates that GRP78 and caspase-7 interact, the invention is not limited to a direct interaction between the two proteins. It is understood that the invention encompasses a cytosolic component that is a complex of polypeptides, including caspase-7, or caspase-7 individually. By preventing the interaction of GRP78 with caspase-7, the agent modulates apoptosis by promoting apoptosis. Alternatively, by promoting the interaction of GRP78 with caspase-7, an agent would modulate apoptosis by inhibiting apoptosis.

In one embodiment, the invention provides a method of modulating apoptosis by contacting a GRP (e.g., GRP74, 78, and/or 94) with an agent that regulates the interaction of the GRP protein with a cytosolic component that mediates apoptosis. As used herein, the term “interact” includes any detectable interactions between molecules. The term “interact” is also meant to include “binding” interactions between molecules. Interactions can, for example, be protein-protein, protein-nucleic acid, and nucleic acid-nucleic acid in nature including hydrogen-bond interactions, covalent-bond interactions and the like.

An “agent”, as used herein, can be any molecule including, for example, a polypeptide, an antibody, a nucleic acid (e.g., an antisense, ribozyme, siRNA or the like) or a small molecule. An agent can be a “therapeutic agent” useful for treating disorders associated with cell proliferation including anti-neoplastic agents and anti-inflammatory agents.

The invention provides apoptotic agents (e.g., GRP antagonists) comprising agents that inhibit the anti-apoptotic affect of GRPs (e.g., GRP78). In one aspect of the invention, a small molecule such as dATP is used to prevent the interaction, and/or disrupt the interaction of GRP78 with caspase-7 thereby inhibiting the anti-apoptotic activity of GRP78. In another aspect, agents are provided that inhibit transcription from a GRP (e.g., GRP78) gene. For example, versipelostatin (VST) is useful to inhibit transcription from GRP78 (Park et al., J. Nat. Canc. Inst., 96(17):1300-1310, 2004; the disclosure of which is incorporated herein by reference). In another aspect, inhibitory nucleic acid molecules (e.g., antisense, ribozymes, siRNA) molecules are used to inhibit the production of GRPs (e.g., GRP78). In one aspect, the apoptotic agents provide a method for inducing apoptosis by inhibiting the production of GRP78 thereby inhibiting the anti-apoptotic affect of GRP78 in a cell. The apoptotic agents of the invention are useful in treating neoplastic and cancer disorders by promoting apoptosis in cells expressing GRPs such as GRP78.

In embodiments where apoptosis is desired, the agent that directly reduces expression/activity of GRP can be a nucleic acid that reduces expression of GRP. In embodiments where anti-apoptotic activity is desired, the nucleic acid can be a sense nucleic acid that encodes a GRP protein (e.g., introduction into a cell can increase the cells GRP activity).

In one embodiment where apoptotic activity is desired an apoptotic agent such as a GRP antagonist is used. In one aspect, an apoptotic nucleic acid agent is used. An apoptotic nucleic acid agent can be an antisense nucleic acid that hybridizes to mRNA encoding a GRP. Antisense nucleic acid molecules for use with the invention are those that specifically hybridize under cellular conditions to cellular mRNA and/or genomic DNA encoding a GRP protein in a manner that inhibits expression of the GRP protein, e.g., by inhibiting transcription and/or translation. The binding may be by conventional base pair complementarity, or, for example, in the case of binding to DNA duplexes, through specific interactions in the major groove of the double helix.

Antisense constructs can be delivered as an expression plasmid which, when transcribed in the cell, produces RNA which is complementary to at least a unique portion of the mRNA and/or endogenous gene which encodes a GRP protein. Alternatively, the antisense construct can take the form of an oligonucleotide probe generated ex vivo which, when introduced into a GRP protein expressing cell, causes inhibition of GRP protein expression by hybridizing with an mRNA and/or genomic DNA coding for a GRP protein. Such antisense molecules may comprise modified nucleotides that are resistant to endogenous nucleases, e.g., exonucleases and/or endonucleases, and are therefore stable in vivo. Additionally, general approaches to constructing oligomers useful in antisense therapy have been reviewed, for example, by Van der Krol et al., Biotechniques 6:958-976, 1988; and Stein et al., Cancer Res. 48:2659-2668, 1988.

Antisense approaches involve the design of nucleic acid molecules (e.g., DNA, RNA, or modified forms thereof) that are complementary to nucleic acids encoding a GRP. The antisense molecules will bind to GRP mRNA transcripts and prevent translation or to the endogenous gene and prevent transcription. Absolute complementarity is not required. The ability to hybridize will depend on both the degree of complementarity and the length of the antisense nucleic acid. Generally, the longer the hybridizing nucleic acid, the more base mismatches it may contain and still form a stable duplex (or triplex, as the case may be). One skilled in the art can ascertain a tolerable degree of mismatch by use of standard procedures to determine the melting point of the hybridized complex.

Antisense nucleic acid molecules that are complementary to the 5′ end of an mRNA, e.g., the 5′ untranslated sequence up to and including the AUG initiation codon, should work most efficiently at inhibiting translation. However, sequences complementary to the 3′ untranslated region of mRNAs have been shown to be effective at inhibiting translation of mRNAs as well. (Wagner, R., Nature 372:333, 1994). Therefore, antisense molecules complementary to either the 5′ or 3′ untranslated, non-coding regions of a GRP mRNA or gene may be used in an antisense approach to inhibit transcription and/or translation of endogenous GRP gene or mRNA, respectively. Oligonucleotides complementary to the 5′ untranslated region of the mRNA should include the complement of the AUG start codon.

The coding strand sequences of GRPs are known. For example, Table 1 provides the coding sequences of some GRPs and related molecular chaperones. Other sequence will be readily apparent and available through GenBank.

TABLE 1

| GRP | GenBank/NCBI Accession No. |
|---|---|
| GRP58 | NM_005313 (SEQ ID NO: 5 and 6) |
| GRP78 | BC020235 and P11021 (SEQ ID NO: 1 and 2) |
| GRP94 | BC066656 and AAH66656 (SEQ ID NO: 15 |
| | and 16) |
| Calreticulin (CALR) | NM_004343 and BT007448 (SEQ ID NO: 7 and 8) |
| calreticulin 3 | NM_145046 (SEQ ID NO: 9 and 10) |
| (CALR3) | |
| PDI | E03087 and NM_006849 (pancreatic) |
| | (SEQ ID NO: 11 and 12) |
| ERp72 | HUMERP72H (SEQ ID NO: 13 and 14) |

Each of the accession numbers and their content is incorporated herein by reference. Given the coding strand sequences encoding, for example, GRP78, antisense nucleic acids can be designed according to the rules of Watson and Crick base pairing. The antisense nucleic acid molecule can be complementary to the entire coding region of a GRP polynucleotide (e.g., GRP78 mRNA), or can be an oligonucleotide, which is antisense to only a portion of the coding or noncoding region of a GRP. For example, the antisense oligonucleotide can be complementary to the region surrounding the transcriptional or translation start site of GRP mRNA. An antisense oligonucleotide can be, for example, about 10, 20, 25, 50, 100, 150, 200, 250, 300, 350, 400 or more nucleotides in length. An antisense nucleic acid molecule can be constructed using chemical synthesis and enzymatic ligation reactions using procedures known in the art. Antisense nucleic acid molecules of the invention may be synthesized by standard methods known in the art, e.g. by use of an automated DNA synthesizer (such as are commercially available from Biosearch, Applied Biosystems). As examples, phosphorothioate oligonucleotides may be synthesized by the method of Stein et al., Nucl. Acids Res. 16:3209, 1988; or methylphosphonate oligonucleotides can be prepared by use of controlled pore glass polymer supports (Sarin et al., Proc. Natl. Acad. Sci. U.S.A. 85:7448-7451, 1988). An antisense nucleic acid molecule can be chemically synthesized using naturally occurring nucleotides or variously modified nucleotides designed to increase the biological stability of the molecules or to increase the physical stability of the duplex formed between the antisense and sense nucleic acids.

The invention includes antisense nucleic acid molecules, which hybridize with a polynucleotide sequence comprising a sequence encoding a GRP. The antisense molecules employed may be unmodified or modified RNA or DNA molecules. Suitable modifications include, but are not limited to, the ethyl or methyl phosphonate modification disclosed in U.S. Pat. No. 4,469,863, the disclosure of which is incorporated by reference, and the phosphorthioate modifications to deoxynucleotides described by LaPlanche, et al., 1986 Nucleic Acids Research, 14:9081, and by Stec, et al., 1984 J. Am. Chem Soc. 106:6077. The modification to the antisense oligonucleotides is typically a terminal modification in the 5′ or 3′ region. Alternatively, the antisense molecules can have chimeric backbones of two or more modified nucleic acid bases, which are modified by different methods. Such methods include, for example, amino acid or nucleic acid modification as described by K. Ramasamy and W. Seifert (Bioorganic and Medicinal Chemistry Letters, 6(15):1799-1804 (1996)) or 4′ sugar substituted olignucleotides described by G. Wang and W. Seifert (Tetrahedron Letters, 37(36):6515-6518 (1996)).

Phosphodiester-linked oligonucleotides are particularly susceptible to the action of nucleases in serum or inside cells, and therefore in a one embodiment the antisense nucleic acid molecules of the invention are phosphorothioate or methyl phosphonate-linked analogues, which have been shown to be nuclease-resistant. Specific examples of some antisense oligonucleotides envisioned for this invention may contain phosphorothioates, phosphotriesters, methyl phosphonates, short chain alkyl or cycloalkyl intersugar linkages or short chain heteroatomic or heterocyclic intersugar (“backbone”) linkages. Typical are phosphorothioates and those with CH₂NHOCH₂, CH₂N(CH₃)OCH₂, CH₂ON(CH₃)CH₂, CH₂N(CH₃)N(CH₃)CH₂ and ON(CH₃)CH₂CH₂ backbones (where phosphodiester is OPOCH₂). Also typical are oligonucleotides having morpholino backbone structures (Summerton and Weller, U.S. Pat. No. 5,034,506). In other embodiments, 2′-methylribonucleotides (Inoue, et al., Nucleic Acids Research, 15:6131, 1987) and chimeric oligonucleotides that are composite RNA-DNA analogues (Inoue, et al., FEBS Lett., 215:327, 1987) may also be used for the purposes described herein. Finally, DNA analogues, such as peptide nucleic acids (PNA) are also included (Egholm, et al., Nature 365:566, 1993; Nielsen et al., Science, 254:1497, 1991) can be used according to the invention. Other oligonucleotides may contain alkyl and halogen-substituted sugar moieties comprising one of the following at the 2′ position: OH, SH, SCH₃, F, OCN, OCH₃OCH₃, OCH₃O(CH₂)nCH₃, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O, S, or N-alkyl; O, S or N alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a cholesteryl group; a conjugate; a reporter group; an intercalator; a group for improving the pharmacokinetic properties of a oligonucleotide; or a group for improving the pharmacodynamic properties of a oligonucleotide and other substituents having similar properties. Oligonucleotides may also have sugar mimetics such as cyclobutyls in place of the pentofuranosyl group. Other embodiments may include at least one modified base form or “universal base” such as inosine. The preparation of base-modified nucleosides, and the synthesis of modified oligonucleotides using said base-modified nucleosides as precursors, has been described, for example, in U.S. Pat. Nos. 4,948,882 and 5,093,232. These base-modified nucleosides have been designed so that they can be incorporated by chemical synthesis into either terminal or internal positions of a oligonucleotide. Such base-modified nucleosides, present at either terminal or internal positions of a oligonucleotide, can serve as sites for attachment of a peptide or other antigen. Nucleosides modified in their sugar moiety have also been described (e.g., U.S. Pat. No. 5,118,802 and U.S. Pat. No. 5,681,940, both of which are incorporated by reference) and can be used similarly. Persons of ordinary skill in this art will be able to select other linkages for use in the invention. These modifications also may be designed to improve the cellular uptake and stability of the oligonucleotides. It is understood that depending on the route or form of administration of the antisense oligonucleotides of the invention, the modification or site of modification will vary (e.g., 5′ or 3′ modification). One of skill in the art can readily determine the appropriate modification without undue experimentation.

Examples of modified nucleotides which can be used to generate the antisense molecules include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense molecule can be produced biologically using an expression vector into which a GRP polynucleotide or fragment thereof has been subcloned in an antisense orientation (i.e., RNA transcribed from the inserted nucleic acid will be of an antisense orientation to a target nucleic acid of interest).

In a specific embodiment, the antisense molecule comprises a sequence as set forth in SEQ ID NO:3 or a fragment thereof. Thus, in one aspect, the antisense molecule includes (i) SEQ ID NO:3; (ii) fragments of SEQ ID NO:3 which inhibit the production of GRP78; (iii) SEQ ID NO:3 or a fragment thereof wherein T is replaced with U; (iv) SEQ ID NO:3 having a modified backbone; and (v) any of (i)-(iv) capable of interacting with a polynucleotide encoding GRP78. Other specific antisense molecules include antisense fragments of nucleic acids encoding GRPs (e.g., fragments of SEQ ID NO:3, antisense fragments of GRP94 and the like; see Table 1).

Small double stranded nucleic acid molecules that can silence a GRP are also provided as part of the invention. Small interfering RNA (siRNA) molecules are provided that interfere with RNA transcription. RNA interference (RNAi) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect persists for multiple cell divisions before gene expression is regained. RNAi is therefore an extremely powerful method for making targeted knockouts or “knockdowns” at the RNA level. RNAi has proven successful in human cells, including human embryonic kidney and HeLa cells (see, e.g., Elbashir et al., Nature, 411(6836):494-8, 2001). In one embodiment, GRP (e.g., GPR78) silencing can be induced in mammalian cells by enforcing endogenous expression of RNA hairpins (see Paddison et al., PNAS USA 99:1443-1448, 2002). In another embodiment, transfection of small (21-23 nt) dsRNA specifically inhibits gene expression (reviewed in Caplen, Trends in Biotechnology 20:49-51, 2002).

Briefly, dsRNA corresponding to a portion of a GRP gene to be silenced is introduced into a cell. The dsRNA can be longer sequences that are subsequently digested into 21-23 nucleotide siRNAs, or short interfering RNAs, or the 21-23 nucleotide siRNA molecules may be directly provided to the cell. The siRNA duplexes bind to a nuclease complex to form what is known as the RNA-induced silencing complex, or RISC. The RISC targets the homologous transcript by base pairing interactions between one of the siRNA strands and the endogenous mRNA. It then cleaves the mRNA about 12 nucleotides from the 3′ terminus of the siRNA (reviewed in Sharp et al., Genes Dev 15: 485-490, 2001; and Hammond et al., Nature Rev Gen 2: 110-119, 2001).

RNAi technology in gene silencing utilizes standard molecular biology methods. dsRNA corresponding to the sequence from a target gene to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Gene silencing effects similar to those of RNAi have been reported in mammalian cells with transfection of a mRNA-cDNA hybrid construct (Lin et al., Biochem Biophys Res Commun, 281(3):639-44, 2001), providing yet another strategy for gene silencing.

Accordingly, the invention provides small interfering nucleic acids (siNA) that interact with a polynucleotide encoding a GRP. In one aspect, the invention provides siNA comprising (i) a sequence as set forth in SEQ ID NO:1, 5, 7, 9, 11, 13, or 15 as set forth in Table 1, and their complement that is 21-23 nucleotides in length and comprises an AA dinucleotide at the 5′ end and a GC content of 30-50%; (ii) a double stranded nucleic acid comprising 5′-AAGGTTACCCATGCAGTTGTT-3′ (SEQ ID NO:4) and its complement; (iii) a sequence as set forth in SEQ ID NO:4 and its complement wherein T is replaced with U; and any of (i)-(iii) wherein the siNA has a modified backbone as described above.

Ribozyme molecules designed to catalytically cleave GRP mRNA transcripts can also be used to prevent translation of GRP mRNA and expression of GRP protein (see, e.g., PCT Publication No. WO 90/11364, published Oct. 4, 1990; Sarver et al., Science 247:1222-1225, 1990 and U.S. Pat. No. 5,093,246). While ribozymes that cleave mRNA at site specific recognition sequences can be used to destroy GRP mRNAs, the use of hammerhead ribozymes is typical. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591, 1988. Typically the ribozyme is engineered so that the cleavage recognition site is located near the 5′ end of GRP mRNA; i.e., to increase efficiency and minimize the intracellular accumulation of non-functional mRNA transcripts. Ribozymes within the invention can be delivered to a cell using a vector.

Endogenous GRP gene expression can also be reduced by inactivating or “knocking out” the GRP gene or its promoter using targeted homologous recombination. See, e.g, Kempin et al., Nature 389: 802 (1997); Smithies et al., Nature 317:230-234, 1985; Thomas and Capecchi, Cell 51:503-512, 1987; and Thompson et al., Cell 5:313-321, 1989. For example, a mutant, non-functional GRP gene variant (or a completely unrelated DNA sequence) flanked by DNA homologous to the endogenous GRP gene (either the coding regions or regulatory regions of the GRP gene) can be used, with or without a selectable marker and/or a negative selectable marker, to transfect cells that express GRP protein in vivo.

The nucleic acids, ribozyme, RNAi, and triple helix molecules used in the invention may be prepared by any method known in the art for the synthesis of DNA and RNA molecules. These include techniques for chemically synthesizing oligodeoxyribonucleotides and oligoribonucleotides well known in the art such as for example solid phase phosphoramide chemical synthesis. Alternatively, RNA molecules may be generated by in vitro and in vivo transcription of DNA sequences encoding the nucleic acid molecule. Such DNA sequences may be incorporated into a wide variety of vectors, which incorporate suitable RNA polymerase promoters. Alternatively, antisense cDNA constructs that synthesize antisense RNA constitutively or inducibly, depending on the promoter used, can be introduced stably into cell lines.

In yet another aspect, the invention provides polypeptide antagonists of GRP activity. Such polypeptides include antibodies, soluble domains of a GRP and polypeptides that interact with a transmembrane domain of a GRP to prevent incorporation of the GRP into a membrane of a cell. For example, as described herein, GRP78 comprises a cytosolic domain and transmembrane domains. The cytosolic domain (e.g., a soluble domain) interacts with cytosolic proteins that induce apoptotis. By inhibiting the interaction of the GRP with the cytosolic proteins that induce apoptosis, the anti-apoptotic effect of the GRP can be inhibited. Furthermore, GRPs (e.g., GRP78 comprises hydrophobic transmembrane domain(s). For example, hydrophobic transmembrane domain III (amino acids 210-260 of SEQ ID NO:2) and/or domain IV (amino acids 400-450 of SEQ ID NO:2) of the protein of GRP78 are useful targets. Polypeptide agents that regulate the ability of a GRP polypeptide to integrate in to the membrane are candidates for modulating apoptosis. For example, a polypeptide agent that inhibits the ability of GRP78 to integrate into the membrane will also be capable of promoting apoptosis because it will prevent GRP78 from interacting with cytosolic components that are required to promote apoptosis. Thus, variants and fragments of a GRP protein (e.g., fragments, analogs and derivatives of native GRP proteins) may also be used in methods of the invention to inhibit anti-apoptotic activity of a GRP (e.g., a GRP antagonist).

As discussed above, the topology of GRP78 indicates that part of GRP78 is exposed to the cytosol, allowing it to interact with cytosolic components. Thus, in another embodiment, the invention provides a method of modulating apoptosis by contacting GRP78 with an agent that inhibits or prevents the ability of the protein to integrate in to the membrane of the endoplasmic reticulum. In yet another embodiment, the invention provides a method of identifying an agent that modulates the interaction of GRP78 with a membrane by providing a polypeptide that includes hydrophobic transmembrane domain III (amino acids 210-260 of SEQ ID NO:2) and/or domain IV (amino acids 400-450 of SEQ ID NO:2) of the protein of GRP78. The polypeptide can be contacted with an agent, and the effect of the agent on the interaction can be determined. Agents that regulate the ability of the polypeptide to integrate in to the membrane are candidates for modulating apoptosis. For example, an agent that inhibits the ability of GRP78 to integrate in to the membrane will also be capable of promoting apoptosis because it will prevent GRP78 from interacting with cytosolic components that are required to promote apoptosis. Accordingly, a method of the invention includes modulating apoptosis by regulating the interaction between ER membrane bound (i.e., integrally associated) GRP78 and a cytosolic component that mediates apoptosis, such as, for example, a complex of proteins that includes caspase-7.

In a further embodiment, the invention provides a method of identifying an agent that modulates the interaction of GRP78 with a cytosolic component that mediates apoptosis. The method includes providing GRP78 integrally-associated with a membrane, providing a cytosolic component comprising at least one caspase, providing an agent, contacting the protein with the component and the agent, simultaneously or in succession, and determining the effect of the agent on the interaction of the protein and the component as compared to a control.

The invention provides methods and compositions that are useful to promote apoptosis in a tissue or cell comprising contacting the tissue or cell with an agent that inhibits the anti-apoptotic activity of a GRP (e.g., GRP78). The methods and compositions are useful in treating neoplastic disorders including cancer and tumor growth. The methods and compositions can be used alone or in combination with other neoplastic/cancer therapies. For example, the methods and compositions of the invention can be used in combination with chemotherapeutic drugs such as, but not limited to, 5-fluorouracil (5FU), cytosine arabinoside, cyclophosphamide, cisplatin, carboplatin, doxyrubicin, etoposide, taxol, and alkylating agents. Furthermore, combinations of nucleic acid inhibitors may be used (e.g., a combination of SEQ ID NO:3 and SEQ ID NO:4).

In another aspect, variants and fragments of a GRP protein (e.g., fragments, analogs and derivatives of native GRP proteins) may also be used in methods of the invention. Such variants include, e.g., a polypeptide encoded by a naturally occurring allelic variant of a native GRP polynucleotide, a polypeptide encoded by an alternative splice form of a native GRP polynucleotide, a polypeptide encoded by a homolog of a native GRP polynucleotide, and a polypeptide encoded by a non-naturally occurring variant of a native GRP polynucleotide.

GRP protein variants have a polypeptide sequence that differs from a native GRP protein in one or more amino acids. The peptide sequence of such variants can feature a deletion, addition, or substitution of one or more amino acids of a native GRP polypeptide. Amino acid insertions are typically of about 1 to 4 contiguous amino acids, and deletions are preferably of about 1 to 10 contiguous amino acids. In some applications, variant GRP proteins substantially maintain a native GRP protein functional activity (e.g., ability to mediate anti-apoptotic activity, bind caspase-7 and the like; are agonists). Such functional variants are useful in treating disorders associated with apoptosis, e.g., ischemia and the like, where it is desirable to reduce apoptosis. For other applications, variant GRP proteins lack or feature a significant reduction in a GRP protein functional activity. Where it is desired to retain a functional activity of native GRP protein, a GRP protein variant can be made by expressing nucleic acid molecules that feature silent or conservative changes. Variant GRP proteins with substantial changes in functional activity can be made by expressing nucleic acid molecules that feature less than conservative changes.

GRP protein fragments corresponding to one or more particular motifs and/or domains or to arbitrary sizes, for example, at least 5, 10, 25, 50, 75, 100, 125, 150, or 175 amino acids in length may be utilized in methods of the invention. In addition, fragments can be chemically synthesized using techniques known in the art such as conventional solid phase f-Moc or t-Boc chemistry. For example, a GRP protein used in the methods of the invention may be arbitrarily divided into fragments of desired length with no overlap of the fragments, or divided into overlapping fragments of a desired length. The fragments can be produced (recombinantly or by chemical synthesis) and tested to identify those fragments, which can function as either agonists or antagonists of a native GRP protein.

Methods of the invention may also involve recombinant forms of the GRP proteins. Recombinant polypeptides, in addition to native GRP protein, are encoded by a nucleic acid that has at least 85% sequence identity (e.g., 85, 86, 87, 88, 89, 90, 91, 92, 93, 94, 95, 96, 97, 98, 99, 100%) with a native GRP nucleic acid sequence. In a one embodiment, variant GRP proteins lack one or more functional activities of a native GRP protein.

GRP protein variants can be generated through various techniques known in the art. For example, GRP protein variants can be made by mutagenesis, such as by introducing discrete point mutation(s), or by truncation. Mutation can give rise to a GRP protein variant having substantially the same, or merely a subset of the functional activity of a native GRP protein. Alternatively, antagonistic forms of the protein can be generated which are able to inhibit the function of the naturally occurring form of the protein, such as by competitively binding to another molecule that interacts with GRP protein (e.g., interferes with the interaction of GRP78 and caspase-7). In addition, agonistic forms of the protein may be generated that constitutively express one or more GRP functional activities. Other variants of GRP proteins that can be generated include those that are resistant to proteolytic cleavage, as for example, due to mutations that alter protease target sequences. Whether a change in the amino acid sequence of a peptide results in a GRP protein variant having one or more functional activities of a native GRP protein can be readily determined by testing the variant for a native GRP protein functional activity.

Nucleic acid molecules encoding GRP fusion proteins may be used in methods of the invention. Such nucleic acids can be made by preparing a construct (e.g., an expression vector) that expresses a GRP fusion protein when introduced into a suitable host. For example, such a construct can be made by ligating a first polynucleotide encoding a GRP protein fused in frame with a second polynucleotide encoding another protein such that expression of the construct in a suitable expression system yields a fusion protein.

As another example, GRP protein variants can be generated from a degenerate oligonucleotide sequence. Chemical synthesis of a degenerate polynucleotide sequence can be carried out in an automatic DNA synthesizer, and the synthetic polynucleotide then ligated into an appropriate expression vector. The purpose of a degenerate set of polynucleotides is to provide, in one mixture, all of the sequences encoding the desired set of potential GRP protein sequences. The synthesis of degenerate oligonucleotides is well known in the art (see for example, Narang, Tetrahedron 39:3, 1983; Itakura et al., Recombinant DNA, Proc 3rd Cleveland Sympos. Macromolecules, ed. A G Walton, Amsterdam: Elsevier pp 273-289, 1981; Itakura et al., Annu Rev. Biochem. 53:323, 1984; Itakura et al., Science 198:1056, 1984; Ike et al., Nucleic Acid Res. 11:477, 1983. Such techniques have been employed in the directed evolution of other proteins (see, for example, Scott et al., Science 249:386-390, 1990; Roberts et al., Proc. Natl. Acad. Sci. USA 89:2429-2433, 1992; Devlin et al., Science 249: 404-406, 1990; Cwirla et al., Proc. Natl. Acad. Sci. USA 87: 6378-6382, 1990; as well as U.S. Pat. Nos. 5,223,409; 5,198,346; and 5,096,815).

Similarly, a library of coding sequence fragments can be provided for a GRP clone in order to generate a variegated population of GRP polypeptide fragments for screening and subsequent selection of fragments having one or more GRP agonist (e.g., anti-apoptotic) or antagonist (e.g., apoptotic) activities. A variety of techniques are known in the art for generating such libraries, including chemical synthesis. In one embodiment, a library of coding sequence fragments can be generated by (i) treating a double-stranded PCR fragment of a GRP polynucleotide coding sequence with a nuclease under conditions wherein nicking occurs only about once per molecule; (ii) denaturing the double-stranded DNA; (iii) renaturing the DNA to form double-stranded DNA which can include sense/antisense pairs from different nicked products; (iv) removing single-stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening products of combinatorial libraries made by point mutations or truncation, and for screening cDNA libraries for products having a certain property. Such techniques will be generally adaptable for rapid screening of the gene libraries generated by the combinatorial mutagenesis of GRP polynucleotide variants. The most widely used techniques for screening large libraries typically involve cloning the library into replicable expression vectors, transforming appropriate cells with the resulting library of vectors, and expressing the combinatorial genes under conditions in which detection of a desired activity facilitates relatively easy isolation of the vector encoding the gene whose product was detected. One screening technique useful to measure anti-apoptotic and apoptotic effects includes determining cell survival in the presence of etoposide. Recombinant products that inhibit (e.g., are antagonistic of) native GRP function will show an increase in cell-death in the presence of etoposide, whereas products that promote (e.g., are agonists of) GRP function will show a reduced cell-death compared to controls. Thus, the invention provides methods of mutagenizing and screening gene products to determine their agonistic and/or antagonistic effect on GRP activity. Agents having agonistic effects are useful for treating ischemia and related disorders that cause unwanted cell death. Agents that have antagonistic effects are useful to treat diseases and disorders having unwanted cell growth (e.g., cell proliferative disorders associated with cancer and the like).

Methods of the invention may utilize mimetics, e.g. peptide or non-peptide agents, that are able to disrupt binding of a GRP protein to other proteins or molecules with which a native GRP protein interacts (e.g., caspase-7). Thus, the mutagenic techniques described herein can also be used to map which determinants of GRP protein participate in the intermolecular interactions involved in, for example, binding of a GRP protein to other proteins which function to carry out apoptosis. Although the invention described thus far has focused on methods and compositions useful to promote apoptosis by inhibiting anti-apoptotic GRPs, the invention also includes methods and compositions that promote anti-apoptotic activity (e.g., in response ischemic injury and the like).

The invention also provides methods and compositions, which promote anti-apoptotic activity of GRPs (e.g., GRP78 and 94). The compositions and methods of this aspect of the invention are useful to treat tissue damage or potential damage to cells or tissues resulting from, for example, stroke, heart attack, hypoxia, hypoglycemia, brain or spinal cord ischemia, or brain or spinal cord trauma. The methods use agents (including, e.g., small molecules, polypeptides, peptides, and nucleic acids) that promote GRP activity, GRP expression, GRP production, and/or GRP association with polypeptides resulting in an inhibition of apoptosis.

The invention provides methods and compositions for modulating GRP expression and/or activity in a cell. Numerous agents for modulating expression/activity of intracellular proteins such as GRP in a cell are known. Any of these suitable for the particular system being used may be employed. Typical agents for promoting (e.g., agonistic) activity of GRPs include mutant/variant GRP polypeptides or fragments, nucleic acids encoding a functional GRP polypeptide or variant, and small organic or inorganic molecules.

Examples of proteins that can modulate GRP expression and/or activity in a cell include native GRP proteins (e.g., to upregulate activity) or variants thereof that can compete with a native GRP protein for binding ligands such as a caspase (e.g., to downregulate apoptosis). Such protein variants can be generated through various techniques known in the art as described herein. For example, GRP protein variants can be made by mutagenesis, such as by introducing discrete point mutation(s), or by truncation (e.g., of the transmembrane region). Mutation can give rise to a GRP variant or fragment having substantially the same, improved, or merely a subset of the functional activity of a native GRP protein. Agonistic (or superagonistic) forms of the protein may be generated that constitutively express one or more GRP functional activities. Other variants of GRP polypeptides that can be generated include those that are resistant to proteolytic cleavage, as for example, due to mutations which alter protease target sequences. Whether a change in the amino acid sequence of a peptide results in a GRP protein variant having one or more functional activities of a native GRP protein can be readily determined by testing the variant for a native GRP protein functional activity (e.g., modulating apoptosis).

As previously noted, the invention provides a method of inhibiting apoptosis in a tissue by overexpressing GRP78 and/or GRP94 in the targeted tissue. Overexpression of a polypeptide in a target tissue can be accomplished by any method known to the skilled artisan. For example, a nucleic acid sequence encoding GRP78 and/or GRP94 can be incorporated in a nucleic acid construct suitable for expression in a targeted tissue. Generally, the construct will possess the appropriate regulatory sequences for expression in the targeted tissue.

The invention provides methods involving modulating levels of GRP in a cell. The cell may be in vitro or in vivo. Where the cell is in vivo it may be present in an animal subject such as any mammal including humans, rats, mice, cats, dogs, goats, sheep, horses, monkeys, apes, rabbits, cattle, and the like. The animal subject can be in any stage of development including adults, young animals, and neonates. Animal subjects also include those in a fetal stage of development. Target tissues can be any within the animal subject such as liver, kidney, heart (e.g., cardiomyocytes), lungs, components of gastrointestinal tract, pancreas, gall bladder, urinary bladder, skeletal muscle, the central nervous system including the brain, eye, skin, bones, and the like.

Various techniques using viral vectors for the introduction of a GRP nucleic acid (e.g., an inhibitory nucleic acid such as an antisense molecule or a GRP variant) into a cell may be utilized in the methods of the invention. Viral vectors for use in the invention are those that exhibit low toxicity to a host cell and induce production of therapeutically useful quantities of a GRP protein or antisense and/or RNAi nucleic acids in a tissue-specific manner. Viral vector methods and protocols that may be used in the invention are reviewed in Kay et al. Nature Medicine 7:33-40, 2001. The use of specific vectors, including those based on adenoviruses, adeno-associated viruses, herpes viruses, and retroviruses are described in more detail below.

The use of recombinant adenoviruses as gene therapy vectors is discussed in W. C. Russell, Journal of General Virology 81:2573-2604, 2000; and Bramson et al., Curr. Opin. Biotechnol. 6:590-595, 1995. Adenovirus vectors are useful in the invention because they (1) are capable of highly efficient gene expression in target cells and (2) can accommodate a relatively large amount of heterologous (non-viral) DNA. A typical form of recombinant adenovirus is a “helper-dependent” adenovirus vector. Such a vector features, for example, (1) the deletion of all or most viral-coding sequences (those sequences encoding viral proteins), (2) the viral inverted terminal repeats (ITRs) which are sequences required for viral DNA replication, (3) up to 28-32 kb of “exogenous” or “heterologous” sequences (e.g., sequences encoding a GRP protein, a GRP variant, an antisense molecule, or an RNAi molecule), and (4) the viral DNA packaging sequence which is required for packaging of the viral genomes into infectious capsids

Other viral vectors that might be used in the invention are adeno-associated virus (AAV)-based vectors. AAV-based vectors are advantageous because they exhibit high transduction efficiency of target cells and can integrate into the host genome in a site-specific manner. Use of recombinant AAV vectors is discussed in detail in Tal, J., J. Biomed. Sci. 7:279-291, 2000 and Monahan and Samulski, Gene Therapy 7:24-30, 2000. A typical AAV vector comprises a pair of AAV inverted terminal repeats (ITRs) which flank at least one cassette containing a tissue (e.g., heart)- or cell (e.g., cardiomyocyte)-specific promoter operably linked to a GRP nucleic acid. The DNA sequence of the AAV vector, including the ITRs, the promoter and GRP gene may be integrated into the host genome.

The use of herpes simplex virus (HSV)-based vectors is discussed in detail in Cotter and Robertson, Curr. Opin. Mol. Ther. 1:633-644, 1999. HSV vectors deleted of one or more immediate early genes (IE) are advantageous because they are generally non-cytotoxic, persist in a state similar to latency in the host cell, and afford efficient host cell transduction. Recombinant HSV vectors can incorporate approximately 30 kb of heterologous nucleic acid. A typical HSV vector is one that: (1) is engineered from HSV type I, (2) has its IE genes deleted, and (3) contains a tissue-specific promoter operably linked to a GRP nucleic acid (e.g., an antisense, RNAi, GRP variant). HSV amplicon vectors may also be useful in various methods of the invention. Typically, HSV amplicon vectors are approximately 15 kb in length, and possess a viral origin of replication and packaging sequences.

Retroviruses such as C-type retroviruses and lentiviruses are also useful in the invention. For example, retroviral vectors may be based on murine leukemia virus (MLU). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous nucleic acids in place of the viral genes. The heterologous nucleic acids typically comprise a tissue-specific promoter and a GRP nucleic acid.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells. They are also highly efficient at transducing human epithelial cells. Lentiviral vectors for use in the invention may be derived from human and non-human (including SUV) lentiviruses. A typical lentiviral vector includes nucleic acid sequences required for vector propagation as well as a tissue-specific promoter operably linked to a GRP nucleic acid.

A lentiviral vector may be packaged into any suitable lentiviral capsid. The substitution of one particle protein with another from a different virus is referred to as “pseudotyping”. The vector capsid may contain viral envelope proteins from other viruses, including murine leukemia virus (MLU) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN), might also be used in the invention. Use of alphaviruses is described in Lundstrom, K., Intervirology 43:247-257, 2000 and Perri et al., Journal of Virology 74:9802-9807, 2000. Alphavirus vectors typically are constructed in a format known as a replicon. A replicon may contain (1) alphavirus genetic elements required for RNA replication, and (2) a heterologous nucleic acid such as one encoding a GRP nucleic acid.

Recombinant, replication-defective alphavirus vectors are advantageous because they are capable of high-level gene expression, and can infect a wide host cell range. Alphavirus replicons may be targeted to specific cell types by displaying on their virion surface a functional ligand or binding domain that would allow selective binding to target cells expressing a cognate binding partner. Alphavirus replicons may establish latency, and therefore long-term heterologous nucleic acid expression in a host cell. The replicons may also exhibit transient heterologous nucleic acid expression in the host cell. To increase tissue selectivity of the virus and reduce risk not only can such a virus have a targeted ligand on the virion surface, but also the heterologous nucleic acid (e.g., a GRP nucleic acid) can be operably linked to a tissue specific promoter.

In addition to viral vector-based methods, non-viral methods may also be used to introduce a GRP nucleic acid into a host cell. A review of non-viral methods of gene delivery is provided in Nishikawa and Huang, Human Gene Ther. 12:861-870, 2001. A non-viral gene delivery method according to the invention employs plasmid DNA to introduce a GRP nucleic acid into a cell. Plasmid-based gene delivery methods are generally known in the art and are described in references such as Ilan, Y., Curr. Opin. Mol. Ther. 1:116-120, 1999, Wolff, J. A., Neuromuscular Disord. 7:314-318, 1997 and Arztl, Z., Fortbild Qualitatssich 92:681-683, 1998.

Methods involving physical techniques for introducing a GRP nucleic acid into a host cell can be adapted for use in the invention. For example, the particle bombardment method of gene transfer utilizes an Accell device (gene gun) to accelerate DNA-coated microscopic gold particles into a target tissue, e.g., a cancer tissue. See, e.g., Yang et al., Mol. Med. Today 2:476-481 1996 and Davidson et al., Rev. Wound Repair Regen. 6:452-459, 2000. As another example, cell electropermeabilization (also termed cell electroporation) may be employed to deliver GRP nucleic acids into cells. See, e.g., Preat, V., Ann. Pharm. Fr. 59:239-244 2001.

Synthetic gene transfer molecules can be designed to form multimolecular aggregates with plasmid DNA. These aggregates can be designed to bind to a target cell surface in a manner that triggers endocytosis and endosomal membrane disruption. Cationic amphiphiles, including lipopolyamines and cationic lipids, may be used to provide receptor-independent GRP nucleic acid transfer into target cells. In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

DNA microencapsulation may be used to facilitate delivery of a GRP nucleic acid. Microencapsulated gene delivery vehicles may be constructed from low viscosity polymer solutions that are forced to phase invert into fragmented spherical polymer particles when added to appropriate nonsolvents. Methods involving microparticles are discussed in Hsu et al., J. Drug Target 7:313-323, 1999 and Capan et al., Pharm. Res. 16:509-513, 1999.

Protein transduction offers an alternative to gene therapy for the delivery of therapeutic proteins into target cells, and methods involving protein transduction are within the scope of the invention. Protein transduction is the internalization of proteins into a host cell from the external environment. The internalization process relies on a protein or peptide which is able to penetrate the cell membrane. To confer this ability on a normally non-transducing protein, the non-transducing protein can be fused to a transduction-mediating protein such as the antennapedia peptide, the HIV TAT protein transduction domain, or the herpes simplex virus VP22 protein. See Ford et al., Gene Ther. 8:1-4, 2001.

There are two major approaches to getting the nucleic acid (optionally contained in a vector) into a subject's cells; in vivo and ex vivo. For in vivo delivery the nucleic acid is injected directly into the subject, usually at the site where the nucleic acid is needed. For ex vivo treatment, the subject's cells are removed, the nucleic acid is introduced into these isolated cells and the modified cells are administered to the subject either directly or, for example, encapsulated within porous membranes which are implanted into the subject (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, viral vectors and the like. A commonly used vector for ex vivo and in vivo delivery is a viral vector as discussed above.

Host cells can be transfected or transformed with expression or cloning vectors described herein and cultured in conventional nutrient media modified as appropriate for inducing promoters, selecting transformants, or amplifying the nucleic acids encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting.

In addition to prokaryotes, eukaryotic microbes such as filamentous fungi or yeast are suitable cloning or expression hosts. Saccharomyces cerevisiae is a commonly used lower eukaryotic host microorganism. Others include Schizosaccharomyces pombe, Kluyveromyces hosts such as, e.g., K. lactis, K. fragilis, K. bulgaricus, K. wickeramii, K. waltii, K. drosophilarum, K. thermotolerans, and K. marxianus; yarrowia; Pichia pastoris; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger. Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59, 1977); baby hamster kidney cells (BHK, ATCC CCL 10); Chinese hamster ovary cells/-DHFR (CHO, Urlaub et al., Proc. Natl. Acad. Sci. USA 77:4216, 1980); mouse sertoli cells (TM4, Mather, Biol. Reprod. 23:243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68, 1982); MRC 5 cells; FS4 cells; and a human hepatoma line (Hep G2).

Host cells are transformed with the above-described GRP nucleic acid expression or cloning vectors and cultured in conventional nutrient media modified as appropriate for inducing promoters, selecting transformants, or amplifying the nucleic acids encoding the desired sequences.

The GRP nucleic acids (e.g., antisense, RNAi, ribozymes, variants, coding sequences and the like) may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the BDB oligopeptide-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216, 1980. A suitable selection gene for use in yeast is the trp1 gene present in the yeast plasmid YRp7 (Stinchcomb et al., Nature, 282:39, 1979; Kingsman et al., Gene, 7:141, 1979; Tschemper et al., Gene, 10:157, 1980). The trp1 gene provides a selection marker for a mutant strain of yeast lacking the ability to grow in tryptophan.

A variety of well-known techniques can be used to identify polypeptides which specifically bind to, for example, GRP78 and/or caspase-7, and regulate their interactions. Exemplary techniques include mobility shift DNA-binding assays, methylation and uracil interference assays, DNase and hydroxy radical footprinting analysis, fluorescence polarization, and UV crosslinking or chemical cross-linkers. For a general overview, see, e.g., Ausubel (chapter 12, DNA-Protein Interactions). Furthermore, biological assays the measure the agonistic and antagonistic effects of such agents are also provided.

For example, the invention provides a screening assay to determine the GRP agonistic or antagonistic effect an agent may have on a cell. The assay comprises contacting a cell expressing a GRP with an agent suspected to have GRP agonist or antagonist activity. Contacting the cell with a chemotherapeutic agent (e.g., etoposide) and measuring the percent survival of the cell or cells in culture. Where cell survival is increased compared to a control, the agent has agonistic activity, wherein the cell survival is decreased the agent has agonistic activity.

In another embodiment, the invention provides pharmaceutical compositions comprising an agent identified by a method of the invention, and instructions for use of the agent in the treatment of a cell proliferative disorder. For example, an agent identified as regulating the interaction between GRP78 and a cytosolic component, or an agent that regulates the ability of GRP78 to integrate in to a membrane, such as an ER membrane, can be included in a pharmaceutical composition to treat a cell proliferative disorder. The treatment can encompass inhibiting the disorder by promoting apoptosis or by inhibiting apoptosis. For example, the methods of the invention are suitable for use in preventing dividing cells from further replication by promoting apoptosis or in preventing non-dividing cells from destruction by inhibiting apoptosis.

The invention provides methods and compositions for treating a subject having a cell proliferative disorder. The subject can be any mammal, and is preferably a human. The contacting can be in vivo or ex vivo. Methods of administering pharmaceutical compositions are known in the art and include, for example, systemic administration, topical administration, intraperitoneal administration, intra-muscular administration, as well as administration directly at the site of a tumor or cell-proliferative disorder and other routes of administration known in the art.

The pharmaceutical compositions according to the invention may be administered locally or systemically. By “therapeutically effective dose” is meant the quantity of a compound according to the invention necessary to prevent, to cure or at least partially arrest the symptoms of the disease and its complications. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249: 1527, (1990); Gilman et al. (eds.) (1990), each of which is herein incorporated by reference.

As used herein, “administering a therapeutically effective amount” is intended to include methods of giving or applying a pharmaceutical composition of the invention to a subject that allow the composition to perform its intended therapeutic function. The therapeutically effective amounts will vary according to factors such as the degree of infection in a subject, the age, sex, and weight of the individual. Dosage regimen can be adjusted to provide the optimum therapeutic response. For example, several divided doses can be administered daily or the dose can be proportionally reduced as indicated by the exigencies of the therapeutic situation.

As used herein, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

Further, methods of the invention can be performed alone or in conjunction with standard medical treatments currently available for treating a cell proliferative disorder. For example, when a tumor is being treated, it may be preferable to remove the majority of a tumor surgically or by radiation prior to introducing a construct of the invention in to the cells comprising the tumor.

The terms “protein”, “peptide” and “polypeptide” as used herein, describe any chain of amino acids, regardless of length or post-translational modification (for example, glycosylation or phosphorylation). Thus, the terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length, naturally occurring proteins as well as recombinantly or synthetically produced polypeptides that correspond to a full-length naturally occurring protein or to particular domains or portions of a naturally occurring protein. The term also encompasses mature proteins which have an added amino-terminal methionine to facilitate expression in prokaryotic cells.

Polypeptides and peptides can be chemically synthesized using known techniques or produced using known molecular biology techniques. Polypeptides and proteins are encoded in the genome of an organism by nucleic acids in discrete functional units sometimes referred to as “genes”. Nucleic acid molecules, however, can be removed and isolated from their naturally occurring environment and engineered and manipulated using molecular biology techniques. The term “isolated” means altered “by the hand of man” from its natural state; i.e., if it occurs in nature, it has been changed or removed from its original environment, or both. For example, a naturally occurring nucleic acid molecule or a polypeptide naturally present in a living animal in its natural state is not “isolated”, but the same nucleic acid or polypeptide separated from the coexisting materials of its natural state is “isolated”, as the term is employed herein.

“Polynucleotide” or “nucleic acid molecule” refers to a polymeric form of nucleotides at least 10 bases in length. By “isolated nucleic acid” is meant a polynucleotide that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an automatically replicating plasmid or virus; or into the genomic DNA of a prokaryote or eukaryote, or which exists as a separate molecule (e.g., a cDNA) independent of other sequences. The nucleic acid molecules of the invention may comprise ribonucleotides, deoxyribonucleotides, or modified forms of either nucleotide. The term includes single and double stranded forms.

The term nucleic acid molecule(s) or polynucleotide(s) generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions.

In addition, a polynucleotide or nucleic acid molecule as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “nucleic acid molecules” as that term is intended herein.

Nucleic acid molecules comprising an antisense molecule, a siRNA molecule, or encoding a GRP polypeptide and the like, as disclosed herein, can be operatively linked to expression control element(s). “Operatively linked” refers to a juxtaposition wherein the components so described are in a relationship permitting them to function in their intended manner. An expression control element(s) operatively linked to a nucleic acid molecule of the invention is ligated such that transcription of the nucleic acid molecule is achieved under conditions compatible with the expression control element(s). As used herein, the term “expression control element(s)” refers to control domain that regulate the expression of a nucleic acid molecule to which it is operatively linked. Expression control element(s) are operatively linked to a nucleic acid molecules when the expression control element(s) control and regulate the transcription and, as appropriate, translation of the nucleic acid molecule. Thus, expression control element(s) can include appropriate promoters, enhancers, transcription terminators, a start condon (i.e., ATG) in front of a protein-encoding nucleic acid, splicing signals for introns, maintenance of the correct reading frame of that gene to permit proper translation of the mRNA, and stop condons. The term “control element(s)” is intended to include, at a minimum, components whose presence can influence expression, and can also include additional components whose presence is advantageous, for example, leader sequences and fusion partner sequences. Expression control element(s) can include a promoter.

By “promoter” is meant a minimal nucleic acid domain sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters, are included in the invention (see e.g., Bitter et al., Methods in Enzymology 153:516-544, 1987). For example, when cloning in bacterial systems, inducible promoters such as pL of bacteriophage-γ, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the nucleic acids of the invention.

A nucleic acid molecule may be designed to selectively hybridize to a target polynucleotide or oligonucleotide under desired conditions. The phrase “selectively (or specifically) hybridizes to” refers to the binding, duplexing, or hybridizing of a molecule only to a particular nucleotide sequence under stringent hybridization conditions when that sequence is present in a complex mixture.

The phrase “stringent hybridization conditions” refers to conditions under which a nucleic acid molecule will hybridize to its target complementary sequence, typically in a complex mixture of nucleic acids, but to no other sequences. In the context of the invention, stringent conditions comprises hybridization and washing under which nucleotide sequences at least 60% homologous to each other typically remain hybridized to each other. Typically, the conditions are such that sequences at least about 65%-70% or 75% or more homologous to each other typically remain hybridized to each other.

Generally, stringent conditions are selected to be about 5 to 10° C. lower than the thermal melting point (Tₘ) for the specific sequence at a defined ionic strength pH. The Tₘ is the temperature (under defined ionic strength, pH, and nucleic acid concentration) at which 50% of the nucleic acid molecules complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions also may be achieved with the addition of destabilizing agents, for example, formamide.

Exemplary highly stringent hybridization conditions can be as following, for example: 50% formamide, 5×SSC and 1% SDS, incubating at 42° C., or 5×SSC and 1% SDS, incubating at 65° C., with wash in 0.2×SSC and 0.1% SDS at 65° C. Alternative conditions include, for example, conditions at least as stringent as hybridization at 68° C. for 20 hours, followed by washing in 2×SSC, 0.1% SDS, twice for 30 minutes at 55° C. and three times for 15 minutes at 60° C. Another alternative set of conditions is hybridization in 6×SSC at about 45° C., followed by one or more washes in 0.2×SSC, 0.1% SDS at 50-65° C. Exemplary moderately stringent hybridization conditions include a hybridization in a buffer of 40% formamide, 1 M NaCl, 1% SDS at 37° C., and a wash in 1×SSC at 45° C.

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for a neoplastic disorder/cancer if, after receiving a therapeutic amount of a GRP antagonist the subject shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues.

The phrase “non-dividing” cell refers to a cell that does not go through mitosis. Non-dividing cells may be blocked at any point in the cell cycle, (e.g., G0/G1, G1/S, G2/M), as long as the cell is not actively dividing. Examples of pre-existing non-dividing cells in the body include neuronal, muscle, liver, skin, heart, lung, and bone marrow cells, and their derivatives.

By “dividing” cell is meant a cell that undergoes active mitosis, or meiosis. Such dividing cells include stem cells, skin cells (e.g., fibroblasts and keratinocytes), gametes, and other dividing cells known in the art. Of particular interest and encompassed by the term dividing cell are cells having cell proliferative disorders, such as neoplastic cells. The term “cell proliferative disorder” refers to a condition characterized by an abnormal number of cells. The condition can include both hypertrophic (the continual multiplication of cells resulting in an overgrowth of a cell population within a tissue) and hypotrophic (a lack or deficiency of cells within a tissue) cell growth or an excessive influx or migration of cells into an area of a body. The cell populations are not necessarily transformed, tumorigenic or malignant cells, but can include normal cells as well.

Cell proliferative disorders include disorders associated with an overgrowth of connective tissues, such as various fibrotic conditions, including scleroderma, arthritis and liver cirrhosis. Cell proliferative disorders include neoplastic disorders such as head and neck carcinomas. Head and neck carcinomas would include, for example, carcinoma of the mouth, esophagus, throat, larynx, thyroid gland, tongue, lips, salivary glands, nose, paranasal sinuses, nasopharynx, superior nasal vault and sinus tumors, esthesioneuroblastoma, squamous call cancer, malignant melanoma, sinonasal undifferentiated carcinoma (SNUC) or blood neoplasia. Also included are carcinoma's of the regional lymph nodes including cervical lymph nodes, prelaryngeal lymph nodes, pulmonary juxtaesophageal lymph nodes and submandibular lymph nodes (Harrison's Principles of Internal Medicine (eds., Isselbacher, et al., McGraw-Hill, Inc., 13th Edition, pp 1850-1853, 1994). Other cancer types, include, but are not limited to, lung cancer, colon-rectum cancer, breast cancer, prostate cancer, urinary tract cancer, uterine cancer lymphoma, oral cancer, pancreatic cancer, leukemia, melanoma, stomach cancer and ovarian cancer.

Methods involving conventional molecular biology techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Various techniques using polymerase chain reaction (PCR) are described, e.g., in Innis et al., PCR Protocols: A Guide to Methods and Applications, Academic Press: San Diego, 1990. PCR-primer pairs can be derived from known sequences by known techniques such as using computer programs intended for that purpose (e.g., Primer, Version 0.5, 81991, Whitehead Institute for Biomedical Research, Cambridge, Mass.). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

A few diseases and disorders (e.g., ischemia and neoplastic disorders) have been mentioned. However, those of skill in the art will recognize that a variety of diseases and degenerative disorders involve aberrant or disregulated apoptosis, resulting in inappropriate or premature cell death or inappropriate cell proliferation. For example, inhibition of cell death may contribute to disease in the immune system by allowing the persistence of self-reactive B and T cells, which leads to autoimmune disease. Furthermore, the infection by certain viruses may depend on suppression of host cell death by anti-apoptotic viral gene products and inhibition of apoptosis can alter the course (lytic vs. latent) of viral infection.

### EXAMPLES

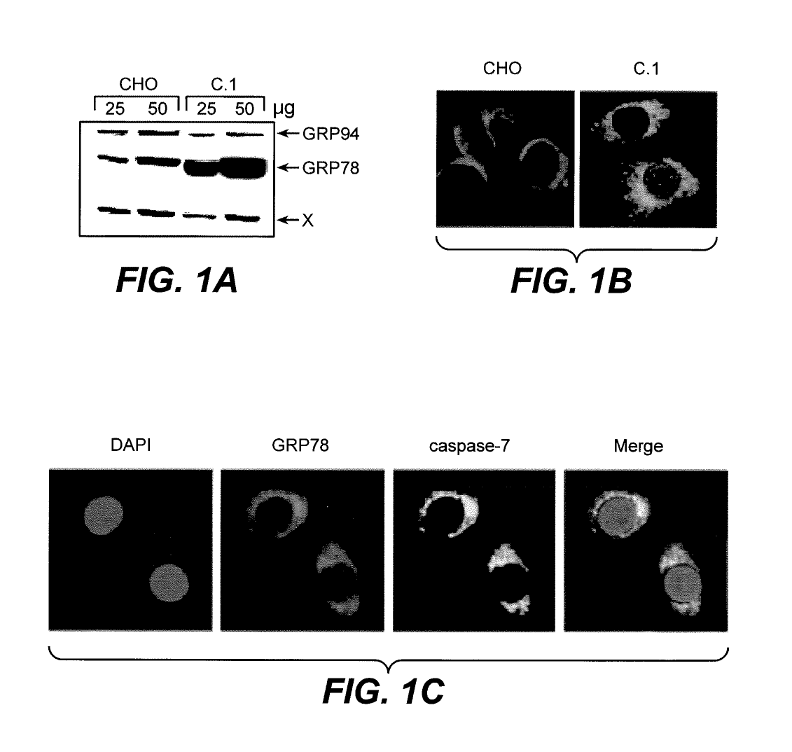

The invention is based, in part, on the discovery that GRPs (e.g., GRP78) confers resistance to topoisomerase inhibitors through protection against drug-induced apoptosis. As shown in FIG. 1, panel A, quantitation of the immunoblots of whole cell extracts showed 5-fold higher GRP78 level in C.1 cells compared with the parental CHO cells, whereas the level of GRP94, also an ER-localized chaperone protein, and a 45-kDa unidentified protein (X) recognizable by the anti-KDEL antibody was relatively constant in both cell lines. In situ immunofluorescence imaging using anti-GRP78 antibody further revealed that in both CHO and C.1 cells, the majority of GRP78 was concentrated in the perinuclear region, consistent with its location in the ER (FIG. 1, Panel B). The intensity of the immunofluorescent signal for GRP78 was greater in the majority of C.1 cells compared with CHO cells.

In examining the distribution of GRP78 and caspase-7 in situ using immunofluorescence, caspase-7 exhibits a perinuclear pattern indicative of ER localization (FIG. 1, Panel C). Confocal microscopy further revealed caspase-7 is in close proximity with a subfraction of GRP78. The co-localization of GRP78 and caspase-7 was primarily detected in the perinuclear/ER region.

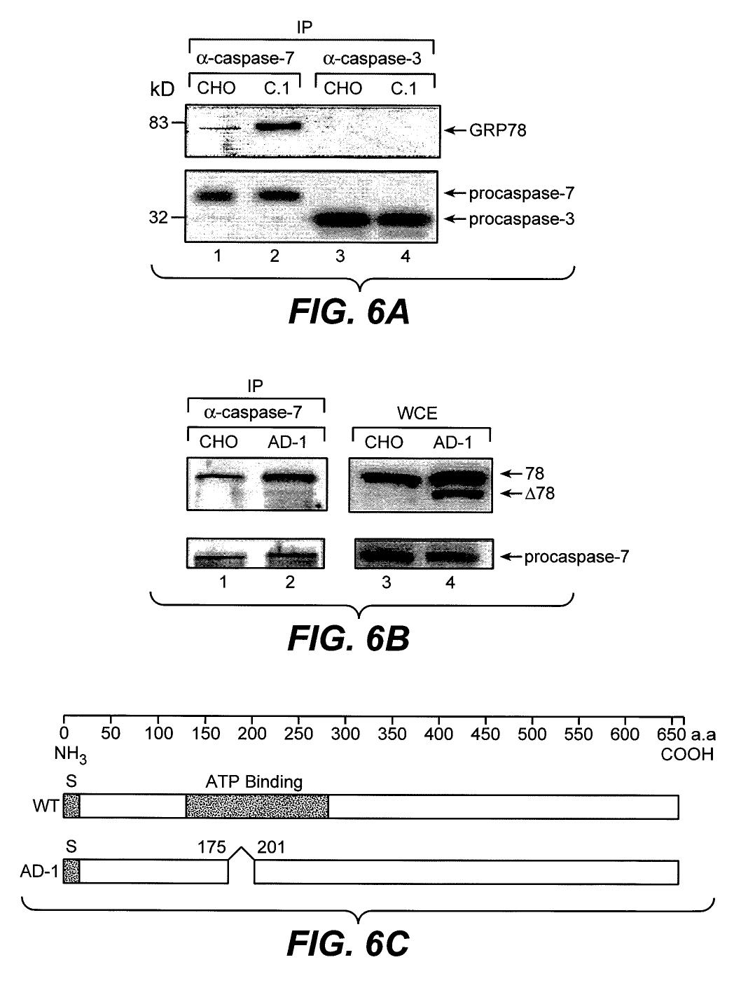

The physical interaction of endogenous caspase-7 with GRP78 was further confirmed using whole cell extracts prepared from CHO and C.1 cells. In agreement with the co-localization results obtained from confocal microscopy, procaspase-7 forms a complex with a high level of GRP78 in C.1 cells (FIG. 6, Panel A, lane 2). For CHO cells, GRP78 was detected as a faint band in the anti-caspase-7 immunoprecipitate using an anti-KDEL antibody (FIG. 6, Panel A, lane 1), and the signal for GRP78 was very enhanced when an anti-hamster GRP78 antibody was used for the Western blots (FIG. 6, Panel B, lane 1). Using anti-caspase-3 as the immunoprecipitating antibody, GRP78 was not detected associated with procaspase-3 in Western blots (FIG. 6, Panel A, lanes 3 and 4). Thus, endogenous GRP78 constitutively associates with procaspase-7. While the data provided herein indicates that GRP78 and caspase-7 interact, the invention is not limited to a direct interaction between the two proteins. It is understood that the invention encompasses a cytosolic component that is a complex of polypeptides, including caspase-7, or caspase-7 individually. By preventing the interaction of GRP78 with caspase, the agent modulates apoptosis by promoting apoptosis. Alternatively, by promoting the interaction of GRP78 with caspase-7, an agent would modulate apoptosis by inhibiting apoptosis.

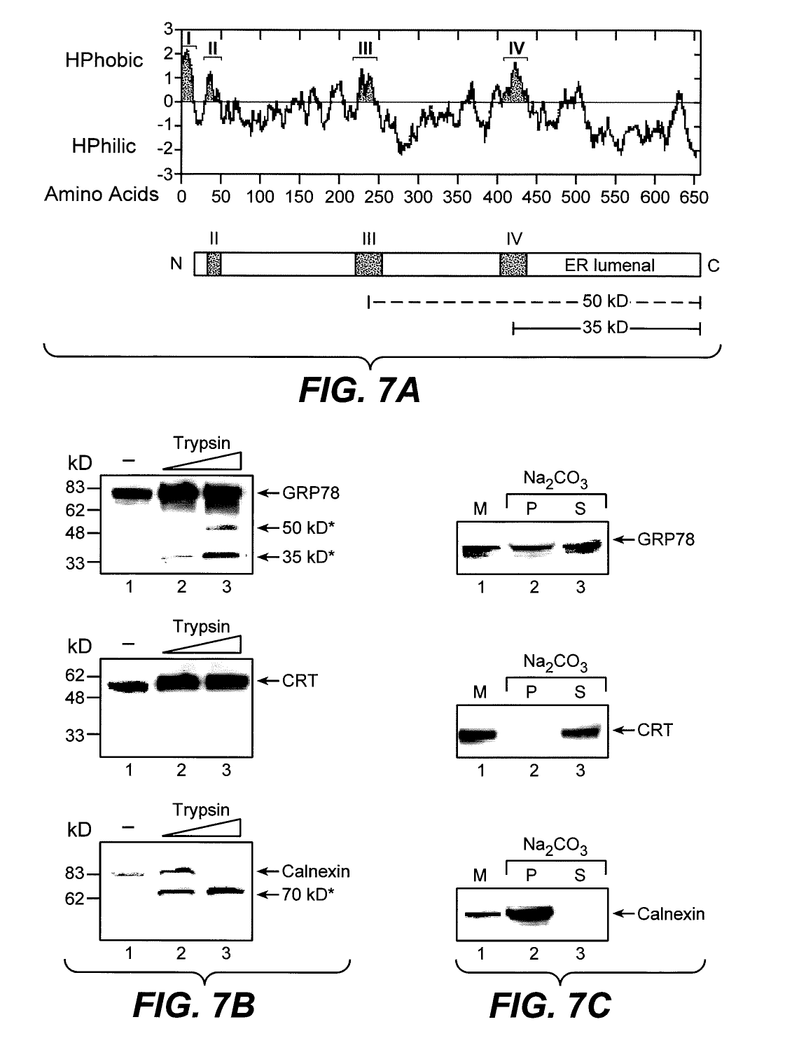

As shown in FIG. 7, Panel A, at low dose of trypsin digestion, a resistant carboxyl band of about 35 kDa was detected. At the higher dose of trypsin, the intensity of the 35-kDa band became stronger, and a minor band of around 50-kDa was also visible. The digestion pattern for the CHO cells was the same, with the resistant bands more prominent for C.1 cells correlating with GRP78 overexpression. The trypsin treatment did not digest ER proteins localized inside the ER lumen as confirmed by the calreticulin control (FIG. 7, Panel B).

In addition, sodium carbonate extraction of the microsome membrane fractions indicates that GRP78 is located in both the membrane and lumenal (FIG. 7, Panel C). These results show that GRP78 is not exclusively an ER lumen protein, rather a subpopulation exist as a transmembrane protein. This is consistent with domains III and IV serving as putative transmembrane domains, with carboxyl fragments locating inside the lumen of the ER rendering them resistant to trypsin digestion (FIG. 7, Panel A). This topology further indicates that part of GRP78 is exposed to the cytosol, allowing it to interact with cytosolic components. Thus, in another embodiment, the invention provides a method of modulating apoptosis by contacting glucose regulated protein 78 (GRP78) with an agent that inhibits or prevents the ability of the protein to integrate in to the membrane of the endoplasmic reticulum. In yet another embodiment, the invention provides a method of identifying an agent that modulates the interaction of glucose regulated protein 78 (GRP78) with a membrane by providing a polypeptide that includes hydrophobic transmembrane domain III (amino acids 210-260 of SEQ ID NO:1 or 2) and/or domain IV (amino acids 400-450 of SEQ ID NO:1 or 2) of the protein of glucose regulated protein 78. The polypeptide can be contacted with an agent, and the effect of the agent on the interaction can be determined. Agents that regulate the ability of the polypeptide to integrate in to the membrane are candidates for modulating apoptosis. For example, an agent that inhibits the ability of GRP78 to integrate in to the membrane will also be capable of promoting apoptosis because it will prevent GRP78 from interacting with cytosolic components that are required to promote apoptosis.

It was further determined that the ATP-binding domain of GRP78 is necessary for the interaction with cytosolic components. The ATP binding domain resides in the amino portion of GRP78 (FIG. 6, Panel C). The invention further encompasses the use of fragments of GRP78 containing the ATP-binding domain in the methods of the invention. For example, a polypeptide that includes amino acids 125-275 of SEQ ID NO:1 or 2, or amino acids 150-250 of SEQ ID NO:1 or 2, or amino acids 175-201 of SEQ ID NO:1 or 2 can be used in a method to identify an agent that regulates the interaction of GRP78 and cytosolic components.

CHO cell line AD-1 that stably expresses a deleted form of GRP78 (FIG. 6, Panel C) was used to determine that the ATP-binding region of GRP78 is necessary for binding to caspase. The deletion spans residues 175 to 201 within the ATP binding domain, resulting in defective ATPase activity. Western blot analysis of whole cell extracts prepared from CHO and AD-1 cells confirmed expression of the deleted GRP78 form, in addition to endogenous wild-type GRP78, in AD-1 cells (FIG. 6B, lanes 3 and 4).

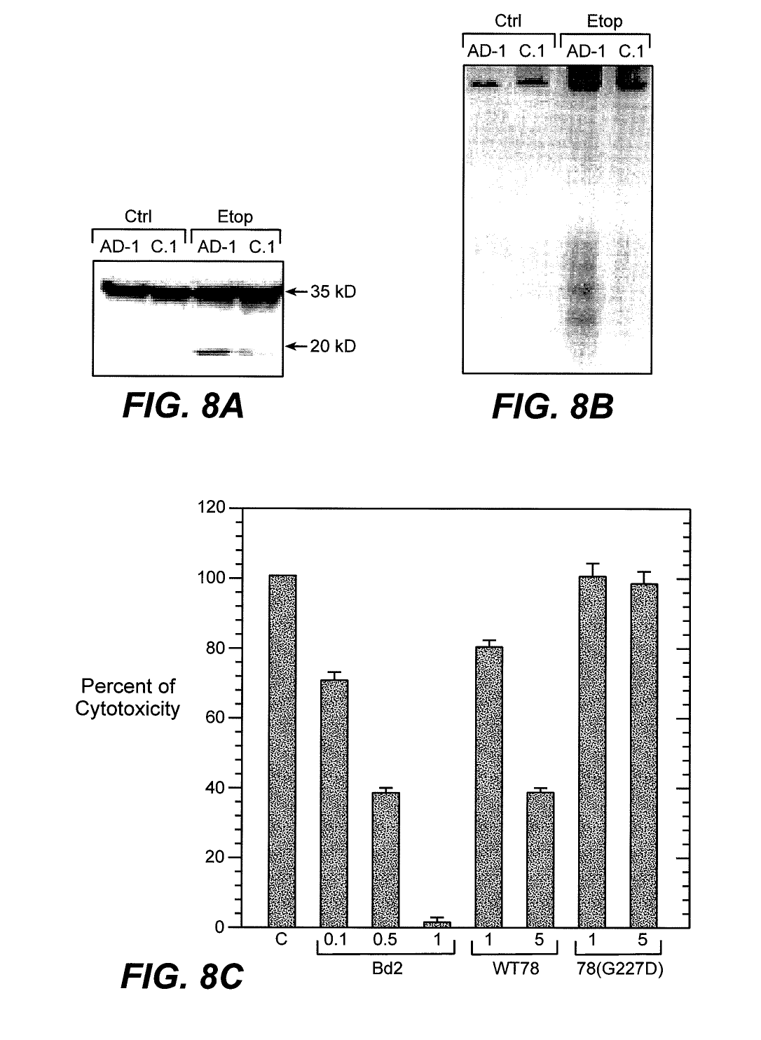

Immunoprecipitation using anti-caspase-7 antibody showed that whereas procaspase-7 is able to form a complex with wild-type GRP78, deletion of residues 175 to 201 abolished its ability to bind to procaspase-7 (FIG. 6, Panel B, lanes 1 and 2. Upon etoposide treatment, AD-1 cells showed more caspase-7 activation in vivo and more extensive DNA fragmentation compared with C.1 cells that overexpress the wild-type protein (FIG. 8, Panels A and B) Annexin labeling and clonogenic survival assays performed with AD-1 cells further showed no protection against etoposide treatment compared with the parental CHO cells.

A transient transfection cell death assay further indicated that inhibition of apoptosis by GRP78 was dependent upon the GRP78-caspase interaction. Cell viability was measured quantitatively by the retention of -galactosidase activity in the cells after drug treatment. As shown in FIG. 8, cells transfected with the expression vector for wild-type GRP78 conferred protection against etoposide treatment. To confirm that the ATP binding function of GRP78 is required for the protective effect of GRP78, the cells were transfected with vectors expressing either wild-type GRP78 or mutant GRP78 (G227D), which carries an amino acid substitution at position 227, destroying its ATP binding ability. As shown in FIG. 8, Panel C, the protective effect was lost with the mutant form of GRP78.

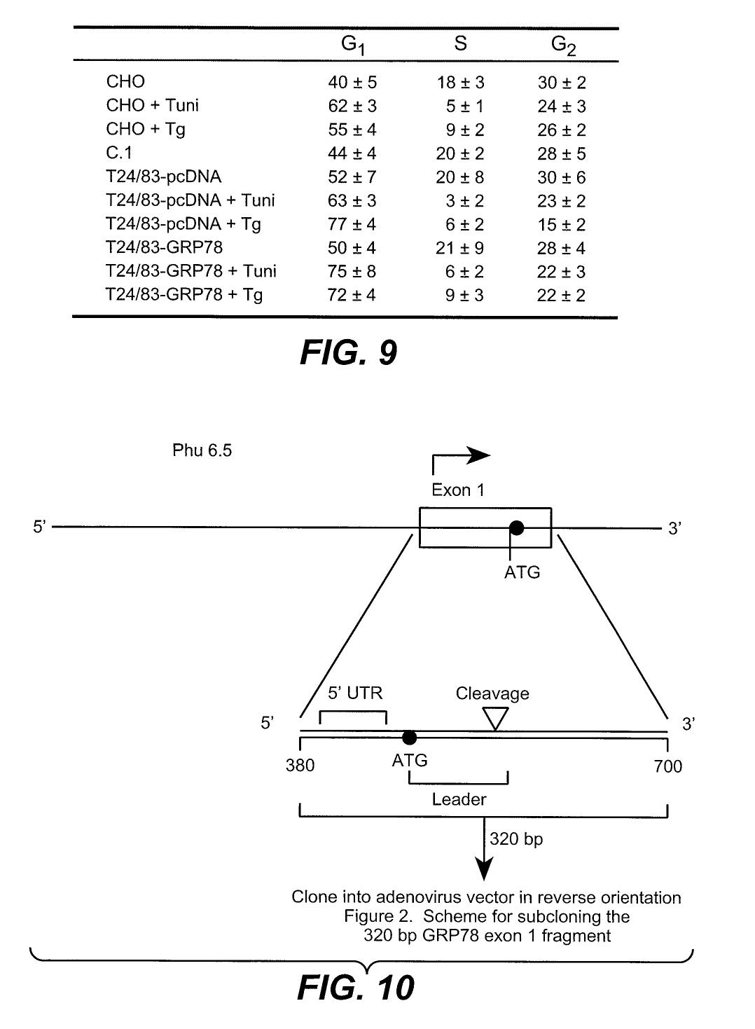

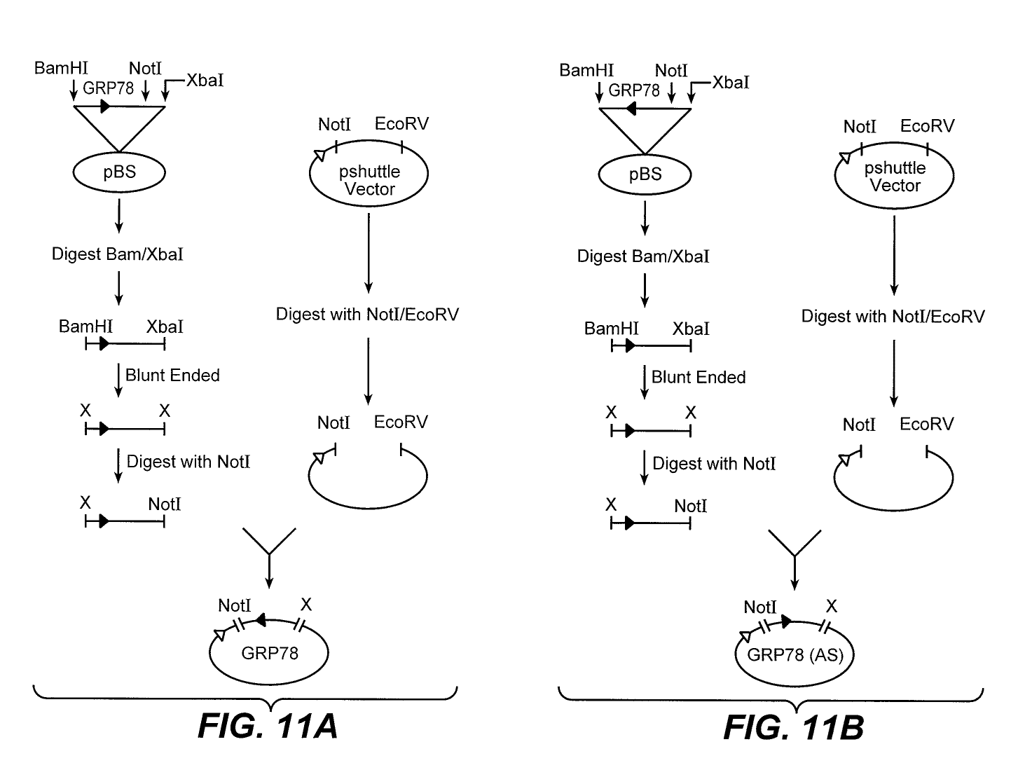

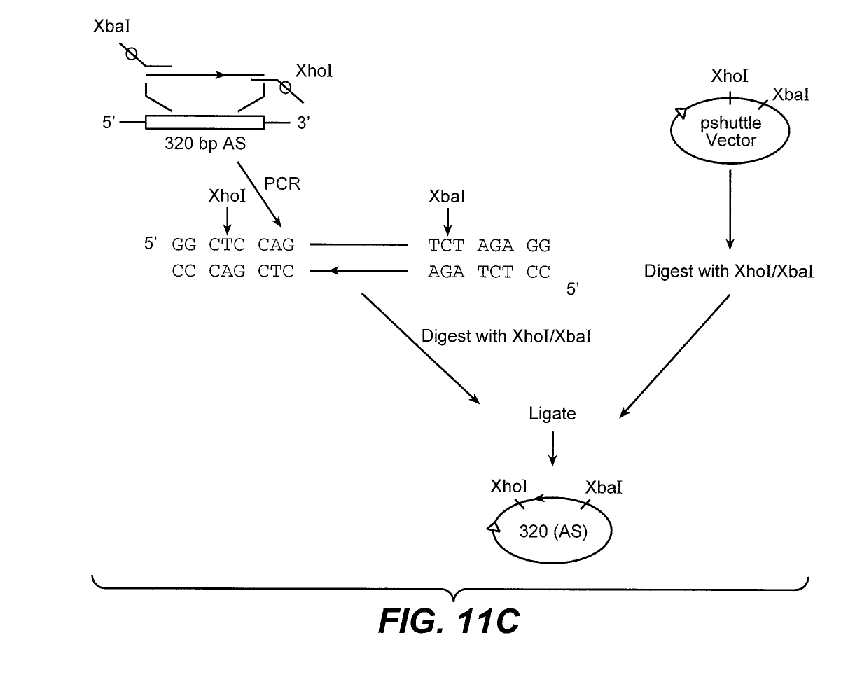

Experiments were performed to look at the effect of GRP78 on chemotherapeutics. A strong cellular promoter (CMV) was used to drive expression of GRP78 in the context of a well-characterized adenovirus vector called pShuttle-CMV. Both the sense and antisense orientation of GRP78 were constructed, to serve the function of overexpression or suppression of GRP78. Two versions of adenovirus with CMV promoter driving antisense (AS) Grp78 were constructed (see FIGS. 11A-C). The construction scheme for the full length AS construct is shown in FIG. 11. An adenovirus construct expressing a partial length AS comprising a 320 bp fragment of the grp78 exon I was cloned in reverse orientation to the CMV promoter (see FIG. 11C). This shorter fragment targets the AUG start codon and may be more effective than the full length antisense molecule.

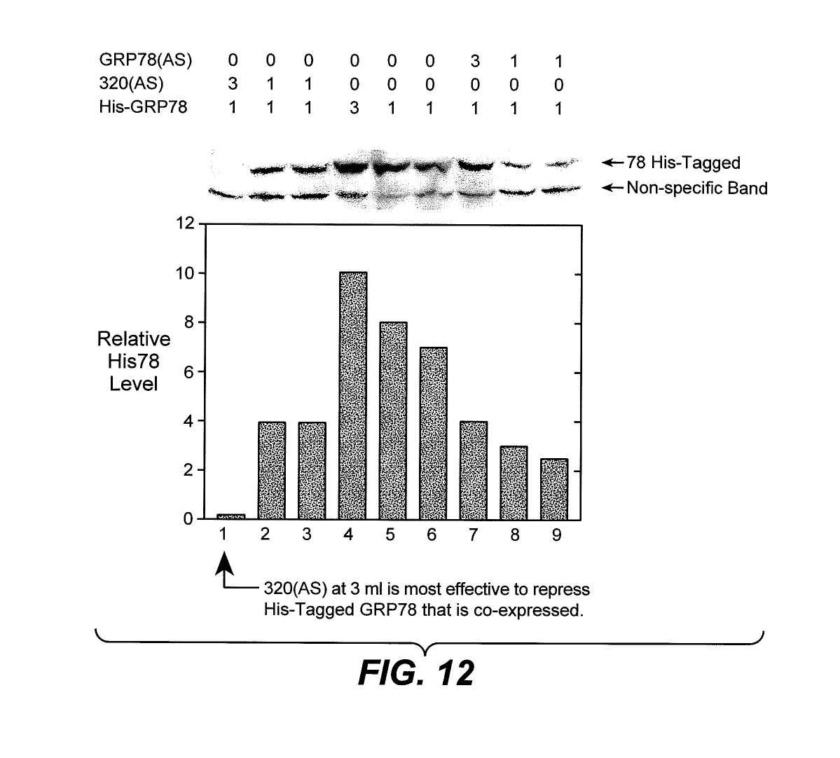

Overexpression of the His-tagged GRP78 in a human 293T tissue culture test system was performed. This cell line was used because it can be infected very efficiently with adenovirus. For this purpose, different doses of adenovirus expressing His-tagged GRP78 was infected into 293T cells. After 72 hr, the cell lysate was prepared and immunoblot was performed to detect the level of His-tagged GRP78, using antibody against the His-tagged which is specific for the adenovirus expressed protein. The results showed that His-tagged GRP78 was expressed in high levels in a dosage dependent manner (FIG. 12, lanes 4, 5 and 6). This proves that the adenovirus construct for expressing full length GRP78 is successful. The next step is to repeat these experiments in MDA and MCF-7 cells. This step is more difficult because the cells are harder to culture and they are more difficult to infect.

As a proof of principle, the two antisense (AS) constructs were tested to determine their ability to suppress the His-tagged GRP78 in the human 293T tissue culture test system. For this purpose, different doses of adenovirus expressing either the full length AS or 320 bp AS was co-infected with adenovirus expressing His-tagged GRP78 into 293T cells. After 72 hr, the cell lysate was prepared and immunoblot was performed to detect the level of His-tagged GRP78, using antibody against the His-tagged which is specific for the adenovirus expressed protein. The results showed that both the full length AS (FIG. 12, lanes 7, 8 and 9) and the 320 bp AS (FIG. 12, lanes 1, 2 and 3) were able to suppress expression of the His-tagged GRP78 in a dosage dependent manner. This proves that the adenovirus constructs for expressing antisense GRP78 are successful. The most drastic reduction was obtained with the 320 bp AS (FIG. 12, lane 1).

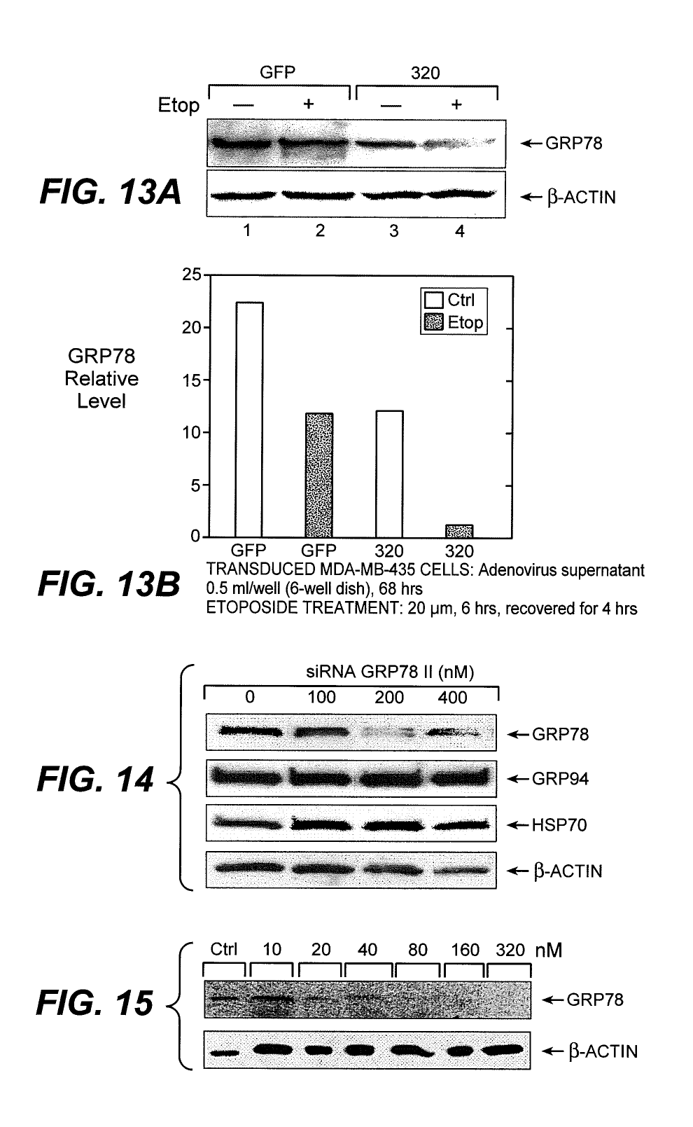

Next human breast cancer MDA-MB-435 cells were infected with the adenovirus expressing the 320 bp AS, in the presence or absence of treatment with the chemotherapy drug etoposide. The same cells were infected with the GFP negative control adenovirus. The results showed that in the mock-infected cells, etoposide treatment by itself reduced the amount of endogenous GRP78 by about 40% (FIG. 13, lanes 1 and 2). Importantly, the 320 by AS construct further reduced GRP78 level significantly, particularly in cells treated with etoposide, such that the final level was less than 10% (FIG. 13, lanes 3 and 4). Thus, both the full length and the 320 bp version of the AS construct targeted against GRP78 blocked expression of GRP78 in a 293T test system. This was repeated in human breast cancer cells and showed that the AS adenovirus is able to suppress endogenous GRP78 expression.

Experiments were also performed to determine the anti-apoptotic effect conferred by overexpression of GRP78. This task was completed. GRP78 overexpression confers resistance to all four drugs (cisplatin, doxorubicin, etoposide and camptothecin). The results are summarized in Table 2.

TABLE 2

% Survival
(Colony Assay)

| | | GRP78 |
|---|---|---|
| | Control | Overexpression |
| Cisplatin (μM) | | |
| 0 | 100 | 100 |
| 3.3 | 43 | 60 |
| 6.6 | 3.7 | 50 |
| 9.9 | 0 | 27 |
| 13.0 | 0 | 16 |
| Doxorubicin (μg/ml) | | |
| 0 | 1 | 100 |
| 0.2 | 80 | 95 |
| 0.4 | 42 | 84 |
| 0.6 | 2 | 68 |
| 0.8 | 0 | 48 |
| 1.0 | 0 | 32 |
| Etoposide (μM) | | |
| 0 | 100 | 100 |
| 0.2 | 71 | 93 |
| 0.4 | 67 | 93 |
| 0.8 | 26 | 77 |
| 1.6 | 1.5 | 63 |
| Camptothecin (ng/ml) | | |
| 0 | 100 | 100 |
| 5 | 68 | 82 |
| 20 | 0.7 | 22 |
| 60 | 0 | 2 |

The antisense approach described above was replaced with an siRNA approach. The sequence of the Grp78 siRNA included:

| | |
|---|---|
| 5′ AAGGTTACCCATGCAGTTGTT 3′ | (SEQ ID NO: 4) |
| 3′ TTCCAATGGGTACGTCAACAA 5′ | (SEQ ID NO: 17) |

When blasted against the human genomic sequence, this sequence is unique and in principle, should not affect any other human gene. To prove this, experiments were performed to test the effect of this siRNA on the expression of GRP78 and related stress proteins GRP94 and HSP70 in human 293T cells. The level of β-actin was used as loading control. As shown in FIG. 14, only GRP78 level is suppressed, confirming that the siRNA is specific for GRP78.

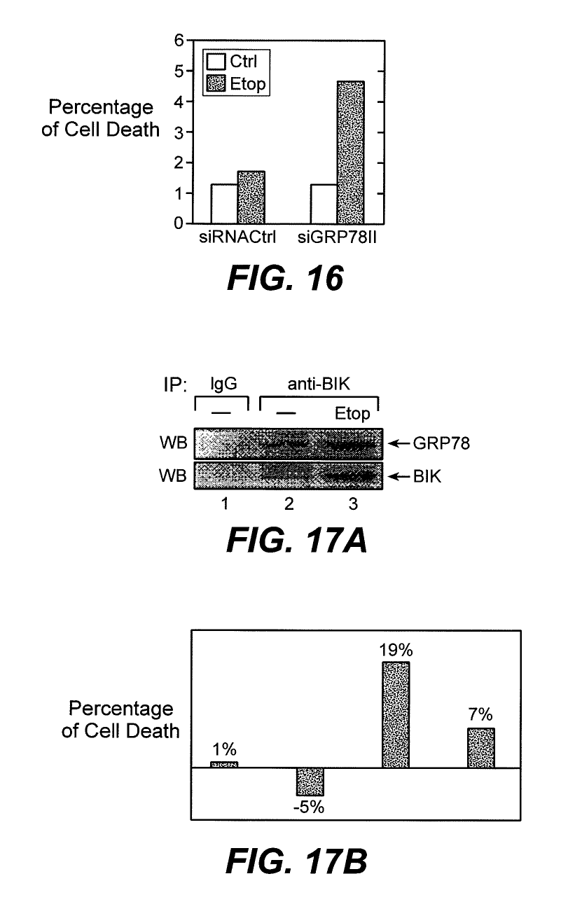

Experiments further showed that Grp78(II) siRNA at 80 nM or higher can significantly suppress GRP78 level in human breast cancer MDA-MB-435 cells (FIG. 15). This translates to more death in the cells treated with Grp78(II) siRNA than control siRNA targeted against the unrelated green fluorescence protein (FIG. 16).

One candidate target of GRP78 action is BIK, the BH3-only protein inducible in response to DNA damage that is located in the ER as well as the mitochondria. Remarkably, ER-targeted BIK can induce cytochrome c release, suggesting it can act at the ER site to initiate a parallel cell death pathway (Germain et al., 2002). To test whether GRP78, as a molecular chaperone, can either directly or indirectly block the action of pro-apoptotic molecules that control the release of cytochrome c from the mitochondria the following experiment was performed. Using 293T as a model system, BIK level was observed to increase following etoposide treatment and co-IP of GRP78 with endogenous BIK (FIG. 17A). These results suggesting that GRP78 overexpression can protect cells from cell death mediated by ER-targeted BIK (FIG. 17B). These findings imply that in cells treated with etoposide, there are both physical and functional interactions between BIK and GPP78. Thus, this can contribute in part to the protective effect of GRP78 towards etoposide-induced cell death.

Cell Culture Conditions—The CHO cells were maintained in -minimum Eagle's medium with nucleosides supplemented with 10% dialyzed fetal calf serum and 1% penicillin/streptomycin/neomycin antibiotics. The C.1 and AD-1 cells were maintained in the above conditions in the presence of 0.1 g/ml methotrexate but without added nucleosides. The establishment of stable T24/83 human transitional bladder carcinoma cell lines overexpressing human GRP78 or transfected with the empty expression vector (pcDNA3.1) has been described. The T24/83 cell lines were maintained in M199 medium supplemented with 10% fetal calf serum containing 1% penicillin/streptomycin/neomycin antibiotics and 200 g/ml G418. The human acute T cell leukemia Jurkat cells were maintained in RPMI 1640 medium supplemented with 10% fetal calf serum containing 1% penicillin/streptomycin/neomycin antibiotics. All the cells were maintained at 37° C. in a humidified atmosphere of 5% CO₂/95% air.

Reagents—Etoposide (Calbiochem) was dissolved in Me₂SO at a concentration of 30 mM and stored at −20° C. Methotrexate (Sigma) was dissolved in a minimum amount of 1 M NaOH, diluted with water to 1 g/ml, and stored at −20° C. Doxorubicin (Bedford Laboratories, Bedford, Ohio) at 2 mg/ml and camptothecin (Amersham Biosciences) at 20 mg/ml were supplied as isotonic solutions.

Cell Cycle Analysis—Following seeding, exponentially growing cells were trypsinized at different days and fixed in 70% ethanol. The fixed cells were treated with PBS containing 0.1% (v/v) Triton X-100, 0.2 mg/ml DNase-free RNase, and 20 μg/ml propidium iodide (PI) for 30 min at room temperature. The cell cycle distributions were analyzed by fluorescence-activated cell sorting (FACS) analysis (FACstar; BD Biosciences). The cell cycle distribution measurements were repeated three to four times.

Clonogenic Survival Assays—Four thousand cells were seeded into 10-cm-diameter dishes. Two days after seeding, cells were treated with etoposide for 6 h, doxorubicin for 1 h, or camptothecin for 24 h at different concentrations as indicated. After drug treatment, the cells were grown in fresh medium for 10 to 14 days. The colonies were washed with ice-cold PBS, fixed with methanol, and stained with 10% Giemsa staining solution. The surviving fraction was determined by dividing the number of the surviving colonies in the drug-treated cells by the number of colonies in the non-treated control groups.

Annexin V Staining and FACS Analysis—CHO, C.1, and T24/83 cells were trypsinized, washed twice with ice-cold PBS, pH 7.4, and resuspended in 1μ binding buffer (10 mM HEPES, pH 7.4, 140 mM NaCl, 2.5 mM CaCl₂ at a concentration of 1×10⁶ cells/ml. One hundred μl of cell suspension was transferred to 5-ml plastic tubes, and 5 μl of annexin V-fluorescein isothiocyanate (PharMingen) and 4 μl of 0.5 mg/ml PI were added. The cells were gently vortexed and incubated in the dark at room temperature for 20 min. Four hundred μl of binding buffer was added to each tube, and annexin V staining was analyzed by flow cytometry within 1 h. Cells negative for both PI and annexin V staining are live cells, annexin V positive staining cells are early apoptotic cells, and PI positive and annexin V positive staining cells are primarily cells in late stages of apoptosis.

Caspase-7 Activation Assays—The cells were either non-treated or treated with 100 μM etoposide for 6 h and harvested after 24 h. The cells were suspended in 5 volumes of a hypotonic buffer (5 mM Tris-HCl, pH 7.4, 5 mM KCl, 1.5 mM MgCl2, 0.1 mM EGTA, pH 8.0, and 1 mM dithiothreitol) in the presence of 2 μg/ml leupeptin, pepstatin, and aprotinin protease inhibitors. After incubation on ice for 20 min, sucrose was added to a final concentration of 250 mM, and the cells were disrupted by douncing eight times in a 1-ml Wheaton Dounce homogenizer. The homogenate was centrifuged twice at 750 μg for 10 min. The supernatant was clarified again at 16,000×g for 15 min at 4° C. and designated as the cytoplasmic fraction. For in vitro caspase-7 activation assay, 150 μg of cell-free extract was incubated with various amounts of cytochrome c and dATP at 37° C. for 1 h. Equal amounts of total proteins were separated, and Western blotting was performed for caspase-7.

Western Blotting—The cell lysate was prepared in radioimmune precipitation assay buffer and subjected to immunoblot with antibodies against GRP78, GRP94, topoisomerase II, caspase-7, and -actin as described. Nitrocellulose membranes containing the transferred proteins were blocked in Tris-buffered saline containing 5% non-fat dry milk and 0.1% Tween 20 for 1 h at room temperature and were probed with the respective primary antibodies. For GRP78, an anti-KDEL mouse monoclonal antibody (SPA-827), an anti-GRP78 rabbit polyclonal antibody directed against the carboxyl ten amino acids of rat GRP78 (SPA-826) (StressGen, Victoria, Canada), or an anti-hamster GRP78 rabbit polyclonal antibody at 1:3000, 1:2000, and 1:5000 dilution, respectively, was used. Dilutions for the other primary antibodies were as follows: anti-calnexin rabbit polyclonal antibody (SPA-865) (StressGen) at 1:2000, anti-calreticulin rabbit polyclonal antibody (SPA-600) (StressGen) at 1:3000, anti- -actin mouse monoclonal antibody (Sigma) at 1:5000, anti-caspase-7 mouse monoclonal antibody (10-1-62) (BD Biosciences) at 1:1000, anti-caspase-3 rabbit polyclonal antibody (Cell Signaling, Beverly, Mass.) at 1:1000, and anti-topoisomerase II mouse monoclonal antibody (SWT3D1) (Oncogene, San Diego, Calif.) at 1:1000. Respective horseradish peroxidase-conjugated secondary antibodies were used, and the protein bands were visualized by the ECL method (Amersham Biosciences).

Transient Transfection Death Assay—Briefly, Jurkat cells were transiently transfected with either CMV-neo-Bcl2 or expression vectors for wild-type hamster GRP78 or a GRP78 ATP-binding site mutant G227D. After drug treatment, cell lysates were prepared and assayed for -galactosidase activity remaining in the surviving cells. The percent cytotoxicity was calculated as described previously.

DNA Fragmentation Assays—The cells were either non-treated or treated with 100 μM etoposide for 12 h and harvested after 48 h. The DNA fragmentation assays were performed using an apoptosis DNA ladder kit (Roche Molecular Biochemicals) according to manufacturer's instructions.

Immunofluorescence Staining and Image Analysis—CHO and C.1 cells were grown to 60% confluence in chamber slides (Nalge Nunc International, Naperville, Ill.), washed twice with PBS, and fixed with 4% paraformaldehyde in PBS for 10 min. The cells were then washed with PBS and permeabilized in PBS containing 0.1% Triton X-100 and 5% bovine serum albumin for 30 min. For detection of GRP78, the cells were stained with a 1:1000 dilution of anti-GRP78 (C-20) goat polyclonal antibody (Santa Cruz Biotechnology, Inc., Santa Cruz, Calif.) and a 1:500 dilution of anti-goat Texas red-conjugated secondary antibody (Vector Labs, Burlingame, Calif.). For detection of caspase-7, the cells were stained with anti-caspase-7 mouse monoclonal antibody (BD Biosciences) at a 1:500 dilution and a 1:500 dilution of anti-mouse fluorescein isothiocyanate-conjugated secondary antibody (Vector Labs). Cells were mounted in Vectashield with DAPI mounting medium (Vector Labs) and visualized on a Zeiss LSM 510 dual-photon confocal microscope. The T24/83 cells were incubated with the same anti-GRP78 polyclonal antibody as described for the CHO cells. Whole cell images were subsequently captured and analyzed using Northern exposure image analysis/archival software (Mississauga, Ontario, Canada).

Co-immunoprecipitation Assays—2×10⁶ cells were lysed in 400 μl of extraction buffer (50 mM Tris-HCl, pH 7.5, 150 mM NaCl, 0.5% Nonidet P-40, and 0.5% deoxycholate, with protease inhibitor tablet (Roche Molecular Biochemicals)) and frozen and thawed three times. 500 μg of total protein extract from each sample was pretreated with 50 μl of protein A-Sepharose beads (Sigma) for 1 h at 4° C. prior to incubation with 5 μg of either anti-caspase-7 mouse monoclonal antibody or anticaspase-3 antibodies for 2 h. Following the incubation period, 50 μl of protein A-Sepharose beads was added, and the mixtures were rotated at 4° C. overnight. The beads were then washed five times with the extraction buffer. The immunoprecipitate was released from the washed beads by the addition of 30 μl of 1× SDS-PAGE sample loading buffer (50 mM Tris-HCl, pH 6.8, 100 mM dithiothreitol, 2% SDS, 0.1% bromphenol blue, 10% glycerol), followed by heating at 100° C. for 10 min. The supernatant obtained after centrifugation was resolved by SDS-PAGE and subjected to Western blot analysis to detect the co-immunoprecipitated proteins.

Isolation of Microsomes and Protease Digestion—The cells were trypsinized, and after washing with cold PBS, were lysed by incubation in 10 volumes of cold hypotonic buffer (10 mM Tris-HCl, pH 7.4), followed by Dounce homogenization. The lysate was immediately adjusted to 0.25 M sucrose, 1 mM MgCl2 and centrifuged at 1000×g for 10 min at 4° C. to remove nuclei and cell debris. The supernatant was further centrifuged at 100,000×g for 90 min. The pellet, representing microsomes, was rinsed briefly with cold water and resuspended in 50 mM Tris-HCl, pH 7.4, and used for proteolytic digestion and sodium-carbonated extraction studies.

Sodium Carbonate Extraction—For separation of ER membranes from lumenal proteins, the microsome pellet was resuspended in 50 volumes of 100 mM sodium carbonate, pH 11.5, and incubated on ice for 1 h. The suspension was then centrifuged for 1 h at 240,000×g at 4° C. The pellet, which represents ER membrane, was rinsed with cold water and resuspended in 1× SDS-PAGE sample loading buffer and analyzed by Western blot. Proteins present in the ER lumen were recovered from the supernatant by the addition of trichloroacetic acid to a final concentration of 10%. The pellet was washed three times with acetone, air-dried, solubilized in the 1× SDS-PAGE sample loading buffer, and analyzed by Western blot.

Limited Tryptic Digestion of Microsomal Proteins—For trypsin digestion reactions, the microsomes were incubated with trypsin (0.01% or 0.05%) for 30 min at room temperature. The proteolytic cleavage reactions were terminated by the addition of 1× SDS-PAGE sample loading buffer and boiling at 100° C. for 5 min. 10-20 μg of total protein from each reaction was analyzed by Western blot.

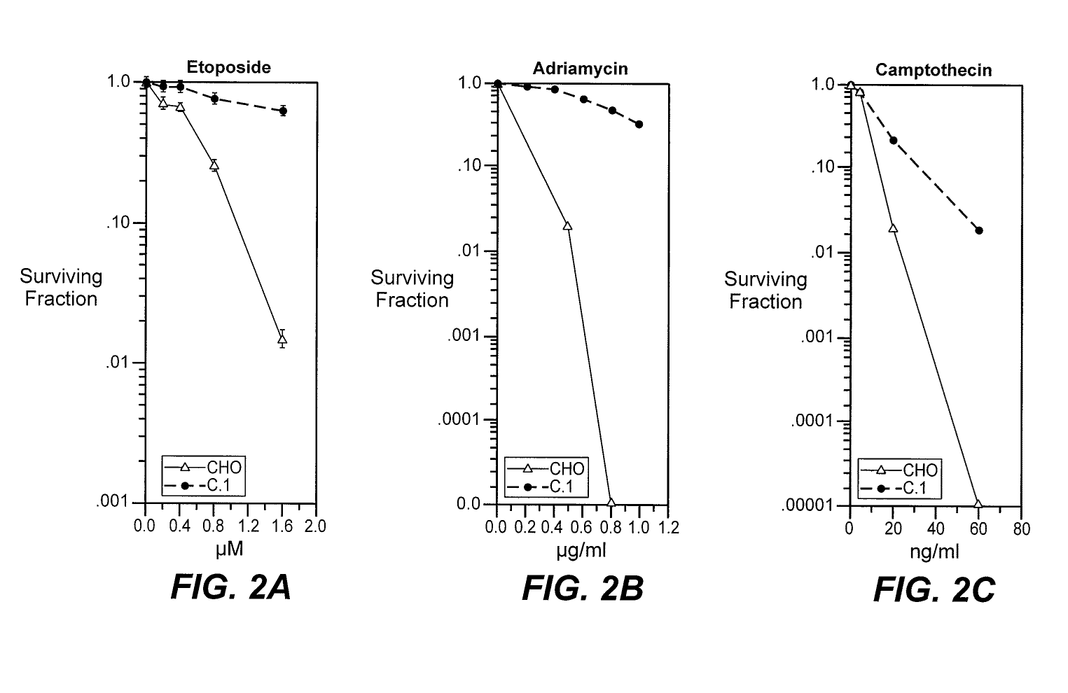

To examine directly whether specific overexpression of GRP78 can lead to the development of drug resistance, CHO and C.1 cells were exposed to various drugs, and cell survival was measured using clonogenic survival assays. Various dosages of etoposide (also referred to as VP16), adriamycin (also referred to as doxorubicin), and camptothecin were tested. Both etoposide and adriamycin are inhibitors of topoisomerase II, and camptothecin is a topoisomerase I inhibitor. The results for each of the drugs tested are shown in FIG. 2. With all three drugs, C.1 cells overexpressing GRP78 conferred higher resistance than CHO cells. These data establish that specific overexpression of GRP78, in the absence of the UPR, is sufficient to render CHO cells more resistant to topoisomerase I and II inhibitors.

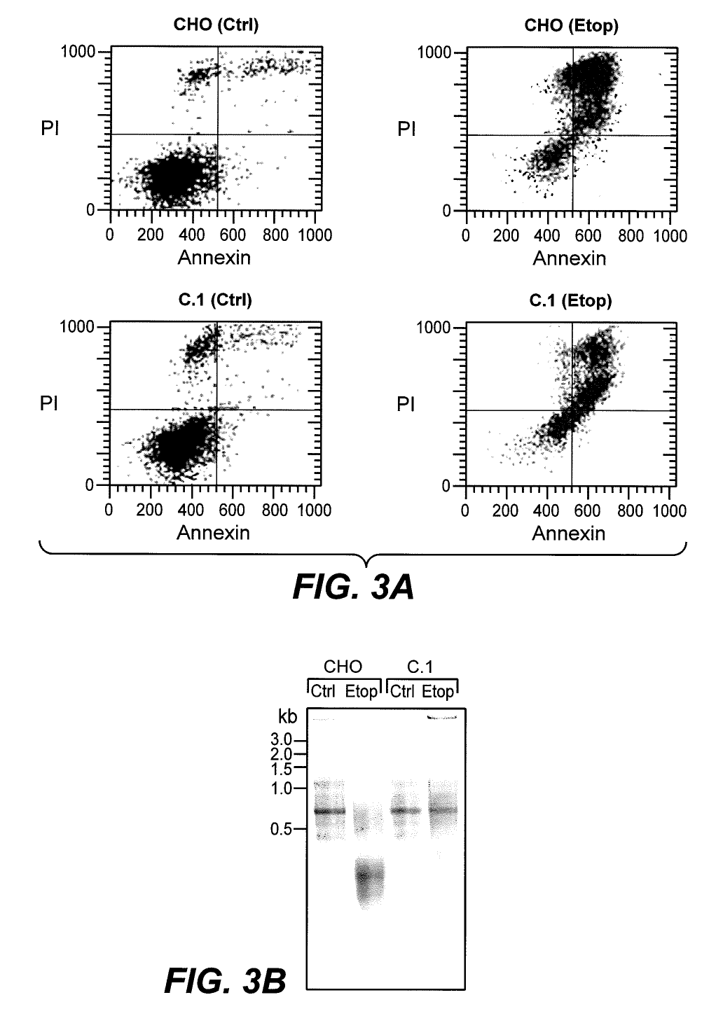

To determine whether GRP78 protects the cells from etoposide-induced apoptosis, CHO and C.1 cells were either nontreated or treated with etoposide and labeled with annexin V and PI. The apoptotic cells were identified by annexin V labeling. For CHO cells, the percentage of apoptotic cells increased 10-fold (from 9 to 90%) upon etoposide treatment; for C.1 cells, the increase was 4.7-fold (from 15 to 70%) (FIG. 3A). More extensive DNA fragmentation was also detected in etoposide-treated CHO but not C.1 cells (Figure, Panel B).

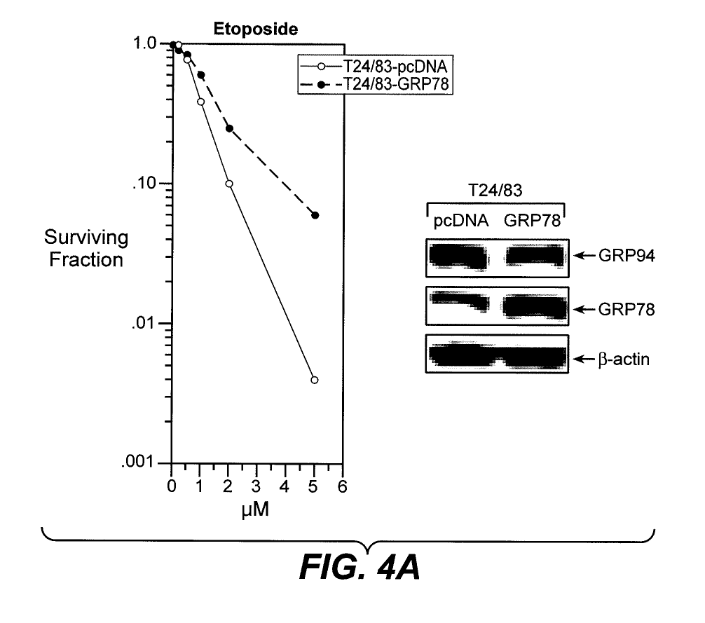

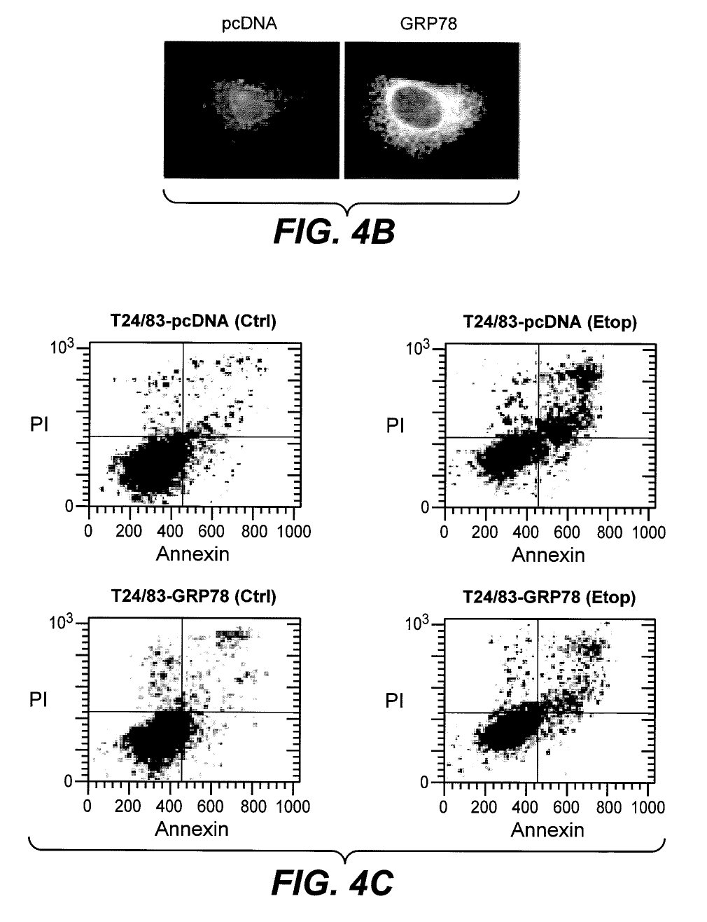

A pair of stably transfected human transitional bladder carcinoma T24/83 cell lines selected and cultured under identical conditions, were used to determine the effect of GRP overexpression on neoplastic cells. The cell line, referred to as T24/83-GRP78, overexpressed human GRP78, and the other line, referred to as T24/83-pcDNA, was stably transfected with the empty expression vector pcDNA (28). Immunoblot analysis followed by normalization against -actin revealed a 3-fold increase in the level of GRP78 expression in the T24/83-GRP78 cells as compared with T24/83-pcDNA cells (FIG. 4A, inset). Overexpression of GRP78 in T24/83-GRP78 cells did not affect the expression level of ER chaperone proteins GRP94, protein disulfide isomerase and calreticulin, or heat shock protein HSP47 (FIG. 4, Panel A). Whole cell imaging revealed much greater GRP78 immunofluorescence for the T24/83-GRP78 cells, confirming the results of the immunoblots (FIG. 4, Panel B).

In agreement with the CHO cell lines, T24/83 cells overexpressing GRP78 exhibited more resistance to etoposide in clonogenic survival assays (FIG. 4, Panel A). Similar protection was observed for adriamycin and camptothecin. For T24/83-cDNA cells, etoposide treatment increased the percentage of annexin V-labeled cells 2.7-fold (from 7 to 19%), as compared with an increase of 1.4-fold (from 8 and 11%) for T24/83-GRP78 cells (FIG. 4C).

With the availability of the GRP78 overexpressing cell lines, the effect of GRP78 overexpression on topoisomerase II level in the absence of an UPR was determined. CHO and C.1 cells were either non-treated or treated with etoposide, and the level of topoisomerase II was determined by immunoblotting (FIG. 5, Panel A). The data indicate that specific GRP78 overexpression has no effect on the topoisomerase II protein level.

Analysis of the cell cycle distribution of exponentially growing cells showed CHO and C.1 cells with similar G1, S, and G2 distribution profiles (FIG. 9). In contrast, CHO cells treated with tunicamycin or thapsigargin, both standard UPR inducers, showed more cells in G1 and a dramatic reduction in S phase cells. A similar pattern was observed for exponentially growing T24/83 cells. In both the vector-transfected and GRP78 overexpressing cells, the percentage of G1, S, and G2 cells is similar. Cells treated with tunicamycin or thapsigargin showed a higher percentage of G1 cells and a lower percentage in S phase (FIG. 9). Collectively, these results show that in contrast to the UPR, specific overexpression of GRP78 does not alter the cell cycle distribution.

A number of embodiments of the invention have been described. Nevertheless, it will be understood that various modifications may be made without departing from the spirit and scope of the invention. Accordingly, other embodiments are within the scope of the following claims.

1. A method of promoting apoptosis in a cell, the method comprising contacting the cell with an agent that inhibits the interaction of glucose regulated protein 78 (GRP78) with a cytosolic component that mediates apoptosis, wherein the agent interacts with the ATP-binding domain of GRP78.

2-4. (canceled)

5. The method of claim 1, wherein the cytosolic component that mediates apoptosis is a caspase.

6. The method claim 5, wherein the caspase is selected from the group consisting of Ced-3, caspase-1, caspase-2, caspase-4, caspase-5, caspase-6, caspase-7, caspase-8, caspase-9, caspase-10, and caspase 11-14.

7. The method of claim 6, wherein the caspase is caspase-7.

8. The method of claim 1, wherein the cytosolic component is a complex of polypeptides.

9-17. (canceled)

18. The method of claim 1, wherein the agent is a small molecule, a protein, a peptide, a peptidomimetic, a nucleic acid molecule or a combination thereof.

19. The method of claim 18, wherein the polypeptide is an antibody.

20. The method of claim 18, wherein the agent is a small molecule.

21-23. (canceled)

24. The method of claim 1, wherein the ATP-binding domain comprises amino acids 125-275 of SEQ ID NO:2.

25. The method of claim 1, wherein the agent interacts with amino acids 150-250 of SEQ ID NO:2.

26. The method of claim 1, wherein the agent interacts with amino acids 175-201 of SEQ ID NO:2.

27-38. (canceled)

39. The method of claim 1, wherein the cell is contacted in vitro.

40. The method of claim 1, wherein the cell is contact in vivo.

41. (canceled)

42. The method of claim 1, wherein the cell is a neoplastic cell.

43-47. (canceled)

48. The method of claim 1, wherein the agent interacts with a hydrophobic transmembrane domain III (amino acids 210-260 of SEQ ID NO:1 or 2) or domain IV (amino acids 400-450 of SEQ ID NO:1 or 2) of GRP78.

49-67. (canceled)

68. A method of modulating apoptosis, the method comprising contacting a cell comprising a caspase polypeptide with an agent that regulates the interaction of the polypeptide with glucose regulated protein 78 (GRP78) endoplasmic reticulum transmembrane protein, wherein the agent interacts with the ATP-binding domain of GRP78.

69. The method claim 68, wherein the caspase is selected from the group consisting of Ced-3, caspase-1, caspase-2, caspase-4, caspase-5, caspase-6, caspase-7, caspase-8, caspase-9, caspase-10, and caspase 11-14.

70. The method of claim 69, wherein the caspase is caspase-7.

71. The method of claim 68, wherein the modulating is by promoting apoptosis.

72-88. (canceled)

89. The method of claim 68, wherein the method further comprises contacting the cell with a chemotherapeutic agent.

90-135. (canceled)

136. The method of claim 68, wherein the agent is an antibody.

137. The method of claim 42, wherein the method further comprises contacting the cell with a chemotherapeutic agent.


SEQUENCE LISTING (17 sequences)

SEQ ID NO: 1 - DNA, 3925 bp, Homo sapiens
  CDS (205)...(2166)
acagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcaagatgaagctctccctggtggccgcgatgctgctgctgctcagcgcggcgcgggccgaggaggaggacaagaaggaggacgtgggcacggtggtcggcatcgacctggggaccacctactcctgcgtcggcgtgttcaagaacggccgcgtggagatcatcgccaacgatcagggcaaccgcatcacgccgtcctatgtcgccttcactcctgaaggggaacgtctgattggcgatgccgccaagaaccagctcacctccaaccccgagaacacggtctttgacgccaagcggctcatcggccgcacgtggaatgacccgtctgtgcagcaggacatcaagttcttgccgttcaaggtggttgaaaagaaaactaaaccatacattcaagttgatattggaggtgggcaaacaaagacatttgctcctgaagaaatttctgccatggttctcactaaaatgaaagaaaccgctgaggcttatttgggaaagaaggttacccatgcagttgttactgtaccagcctattttaatgatgcccaacgccaagcaaccaaagacgctggaactattgctggcctaaatgttatgaggatcatcaacgagcctacggcagctgctattgcttatggcctggataagagggagggggagaagaacatcctggtgtttgacctgggtggcggaaccttcgatgtgtctcttctcaccattgacaatggtgtcttcgaagttgtggccactaatggagatactcatctgggtggagaagactttgaccagcgtgtcatggaacacttcatcaaactgtacaaaaagaagacgggcaaagatgtcaggaaagacaatagagctgtgcagaaactccggcgcgaggtagaaaaggccaaacgggccctgtcttctcagcatcaagcaagaattgaaattgagtccttctatgaaggagaagacttttctgagaccctgactcgggccaaatttgaagagctcaacatggatctgttccggtctactatgaagcccgtccagaaagtgttggaagattctgatttgaagaagtctgatattgatgaaattgttcttgttggtggctcgactcgaattccaaagattcagcaactggttaaagagttcttcaatggcaaggaaccatcccgtggcataaacccagatgaagctgtagcgtatggtgctgctgtccaggctggtgtgctctctggtgatcaagatacaggtgacctggtactgcttgatgtatgtccccttacacttggtattgaaactgtgggaggtgtcatgaccaaactgattccaaggaacacagtggtgcctaccaagaagtctcagatcttttctacagcttctgataatcaaccaactgttacaatcaaggtctatgaaggtgaaagacccctgacaaaagacaatcatcttctgggtacatttgatctgactggaattcctcctgctcctcgtggggtcccacagattgaagtcacctttgagatagatgtgaatggtattcttcgagtgacagctgaagacaagggtacagggaacaaaaataagatcacaatcaccaatgaccagaatcgcctgacacctgaagaaatcgaaaggatggttaatgatgctgagaagtttgctgaggaagacaaaaagctcaaggagcgcattgatactagaaatgagttggaaagctatgcctattctctaaagaatcagattggagataaagaaaagctgggaggtaaactttcctctgaagataaggagaccatggaaaaagctgtagaagaaaagattgaatggctggaaagccaccaagatgctgacattgaagacttcaaagctaagaagaaggaactggaagaaattgttcaaccaattatcagcaaactctatggaagtgcaggccctcccccaactggtgaagaggatacagcagaaaaagatgagttgtagacactgatctgctagtgctgtaatattgtaaatactggactcaggaacttttgttaggaaaaaattgaaagaacttaagtctcgaatgtaattggaatcttcacctcagagtggagttgaaactgctatagcctaagcggctgtttactgcttttcattagcagttgctcacatgtctttgggtgggggggagaagaagaattggccatcttaaaaagcaggtaaaaaacctgggttagggtgtgtgttcaccttcaaaatgttctatttaacaactgggtcatgtgcatctggtgtaggaagttttttctaccataagtgacaccaataaatgtttgttatttacactggtctaatgtttgtgagaagcttctaattagatcaattacttattttaggaaatttaagactagatactcgtgtgtggggtgaggggagggagtatttggtatgttgggataaggaaacacttctatttaatgcttccagggattttttttttttttttttaaccctcctgggcccaagtgatccttccacctcagtctcccagctaattgagaccacaggcttgttaccaccatgctcggcttttgcattaatctaagaaaaggggagagaagttaatccacatctttactcaggcaaggggcatttcacagtgcccaagagtggggttttcttgaacatacttggtttcctatttccccttatctttctaaaactgcctttctggtggctttttttaaaattattactaatgatgcttttatagctgcttggattctctgagaaatgatggggagtgagtgatcactggtattaactttatacacttggatttcatttgtaactttaggatgtaaaggtatattgtgaaccctagctgtgtcagaatctccatccctgaaatttctcattagtggtactggggtgggatcttggatggtgacattgaaactacactaaatcccctcactatgaatgggttgttaaaggcaatggtttgtgtcaaaactggtttaggattacttagattgtgttcctgaagaaaagagtccaggtaaatggtatgatcaataaaggacaggctggtgctaacataaaatccaatattgtaatcctagcactttgggaggccaaggcgggtggatcacaaggtcaagagatagagaccatctttgccaacatggtgaaactccatctctactgaaaatacaaaaattagctgggcgtggtagtgcaagctgaaggctgaggcaggagaatcactcgaacccgggaggcagaggttgcagtgagccgagatcacaccactgtactccagcccggcactccagcctggcgacaagagtgagactccacctcaaaaaaaaaaaaaagaatccaatactgcccaaggataggtattttatagatgggcaactggctgaaaggttaattctctagggctagtagaactggatcccaacaccaaactcttaattagacctaggcctcagctgcactgcccgaaaagcatttgggcagaccctgagcagaatactggtctcaggccaagcccaatacagccattaaagatgacctacagtgctgtgtaccctggggcaatagggttaaatggtagttagcaactagggctagtcttcccttacctcaaaggctctcactaccgtggaccacctagtctgtaactctttctgaggagctgttactgaatattaaaaagatagacttcaaaaaaaaaaaaaaaaaa

SEQ ID NO: 2 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 3 - DNA, 3925 bp, Homo sapiens
ttttttttttttttttttgaagtctatctttttaatattcagtaacagctcctcagaaagagttacagactaggtggtccacggtagtgagagcctttgaggtaagggaagactagccctagttgctaactaccatttaaccctattgccccagggtacacagcactgtaggtcatctttaatggctgtattgggcttggcctgagaccagtattctgctcagggtctgcccaaatgcttttcgggcagtgcagctgaggcctaggtctaattaagagtttggtgttgggatccagttctactagccctagagaattaacctttcagccagttgcccatctataaaatacctatccttgggcagtattggattcttttttttttttttgaggtggagtctcactcttgtcgccaggctggagtgccgggctggagtacagtggtgtgatctcggctcactgcaacctctgcctcccgggttcgagtgattctcctgcctcagccttcagcttgcactaccacgcccagctaatttttgtattttcagtagagatggagtttcaccatgttggcaaagatggtctctatctcttgaccttgtgatccacccgccttggcctcccaaagtgctaggattacaatattggattttatgttagcaccagcctgtcctttattgatcataccatttacctggactcttttcttcaggaacacaatctaagtaatcctaaaccagttttgacacaaaccattgcctttaacaacccattcatagtgaggggatttagtgtagtttcaatgtcaccatccaagatcccaccccagtaccactaatgagaaatttcagggatggagattctgacacagctagggttcacaatatacctttacatcctaaagttacaaatgaaatccaagtgtataaagttaataccagtgatcactcactccccatcatttctcagagaatccaagcagctataaaagcatcattagtaataattttaaaaaaagccaccagaaaggcagttttagaaagataaggggaaataggaaaccaagtatgttcaagaaaaccccactcttgggcactgtgaaatgccccttgcctgagtaaagatgtggattaacttctctccccttttcttagattaatgcaaaagccgagcatggtggtaacaagcctgtggtctcaattagctgggagactgaggtggaaggatcacttgggcccaggagggttaaaaaaaaaaaaaaaaaatccctggaagcattaaatagaagtgtttccttatcccaacataccaaatactccctcccctcaccccacacacgagtatctagtcttaaatttcctaaaataagtaattgatctaattagaagcttctcacaaacattagaccagtgtaaataacaaacatttattggtgtcacttatggtagaaaaaacttcctacaccagatgcacatgacccagttgttaaatagaacattttgaaggtgaacacacaccctaacccaggttttttacctgctttttaagatggccaattcttcttctcccccccacccaaagacatgtgagcaactgctaatgaaaagcagtaaacagccgcttaggctatagcagtttcaactccactctgaggtgaagattccaattacattcgagacttaagttctttcaattttttcctaacaaaagttcctgagtccagtatttacaatattacagcactagcagatcagtgtctacaactcatctttttctgctgtatcctcttcaccagttgggggagggcctgcacttccatagagtttgctgataattggttgaacaatttcttccagttccttcttcttagctttgaagtcttcaatgtcagcatcttggtggctttccagccattcaatcttttcttctacagctttttccatggtctccttatcttcagaggaaagtttacctcccagcttttctttatctccaatctgattctttagagaataggcatagctttccaactcatttctagtatcaatgcgctccttgagctttttgtcttcctcagcaaacttctcagcatcattaaccatcctttcgatttcttcaggtgtcaggcgattctggtcattggtgattgtgatcttatttttgttccctgtacccttgtcttcagctgtcactcgaagaataccattcacatctatctcaaaggtgacttcaatctgtgggaccccacgaggagcaggaggaattccagtcagatcaaatgtacccagaagatgattgtcttttgtcaggggtctttcaccttcatagaccttgattgtaacagttggttgattatcagaagctgtagaaaagatctgagacttcttggtaggcaccactgtgttccttggaatcagtttggtcatgacacctcccacagtttcaataccaagtgtaaggggacatacatcaagcagtaccaggtcacctgtatcttgatcaccagagagcacaccagcctggacagcagcaccatacgctacagcttcatctgggtttatgccacgggatggttccttgccattgaagaactctttaaccagttgctgaatctttggaattcgagtcgagccaccaacaagaacaatttcatcaatatcagacttcttcaaatcagaatcttccaacactttctggacgggcttcatagtagaccggaacagatccatgttgagctcttcaaatttggcccgagtcagggtctcagaaaagtcttctccttcatagaaggactcaatttcaattcttgcttgatgctgagaagacagggcccgtttggccttttctacctcgcgccggagtttctgcacagctctattgtctttcctgacatctttgcccgtcttctttttgtacagtttgatgaagtgttccatgacacgctggtcaaagtcttctccacccagatgagtatctccattagtggccacaacttcgaagacaccattgtcaatggtgagaagagacacatcgaaggttccgccacccaggtcaaacaccaggatgttcttctccccctccctcttatccaggccataagcaatagcagctgccgtaggctcgttgatgatcctcataacatttaggccagcaatagttccagcgtctttggttgcttggcgttgggcatcattaaaataggctggtacagtaacaactgcatgggtaaccttctttcccaaataagcctcagcggtttctttcattttagtgagaaccatggcagaaatttcttcaggagcaaatgtctttgtttgcccacctccaatatcaacttgaatgtatggtttagttttcttttcaaccaccttgaacggcaagaacttgatgtcctgctgcacagacgggtcattccacgtgcggccgatgagccgcttggcgtcaaagaccgtgttctcggggttggaggtgagctggttcttggcggcatcgccaatcagacgttccccttcaggagtgaaggcgacataggacggcgtgatgcggttgccctgatcgttggcgatgatctccacgcggccgttcttgaacacgccgacgcaggagtaggtggtccccaggtcgatgccgaccaccgtgcccacgtcctccttcttgtcctcctcctcggcccgcgccgcgctgagcagcagcagcatcgcggccaccagggagagcttcatcttgccagccagttgggcagcagcaggcagtccagccacaggccgtagcacaggagcacagcgcaatttccgacttgcaggcggcaggggcccggggtcacaaggcgccacgaaccaggcgaagggcaggtctagaaatacaggccgcggcgcttccctctcacactcgcgaaacaccccaataggtcaatctgtctgtgctgt

SEQ ID NO: 4 - DNA, 21 bp, Homo sapiens
aaggttacccatgcagttgtt

SEQ ID NO: 5 - DNA, 2074 bp, Homo sapiens
  CDS (90)...(1604)
caagcagcgggttagtggtcgcgcgcccgacctccgcagtcccagccgagccgcgacccttccggccgtccccaccccacctcgccgccatgcgcctccgccgcctagcgctgttcccgggtgtggcgctgcttcttgccgcggcccgcctcgccgctgcctccgacgtgctagaactcacggacgacaacttcgagagtcgcatctccgacacgggctctgcgggcctcatgctcgtcgagttcttcgccccctggtgtggacactgcaagagacttgcacctgagtatgaagctgcagctaccagattaaaaggaatagtcccattagcaaaggttgattgcactgccaacactaacacctgtaataaatatggagtcagtggatatccaaccctgaagatatttagagatggtgaagaagcaggtgcttatgatggacctaggactgctgatggaattgtcagccacttgaagaagcaggcaggaccagcttcagtgcctctcaggactgaggaagaatttaagaaattcattagtgataaagatgcctctatagtaggttttttcgatgattcattcagtgaggctcactccgagttcctaaaagcagccagcaacttgagggataactaccgatttgcacatacgaatgttgagtctctggtgaacgagtatgatgataatggagagggtatcatcttatttcgtccttcacatctcactaacaagtttgaggacaagactgtggcatatacagagcaaaaaatgaccagtggcaaaattaaaaagtttatccaggaaaacatttttggtatctgccctcacatgacagaagacaataaagatttgatacagggcaaggacttacttattgcttactatgatgtggactatgaaaagaacgctaaaggttccaactactggagaaacagggtaatgatggtggcaaagaaattcctggatgctgggcacaaactcaactttgctgtagctagccgcaaaacctttagccatgaactttctgattttggcttggagagcactgctggagagattcctgttgttgctatcagaactgctaaaggagagaagtttgtcatgcaggaggagttctcgcgtgatgggaaggctctggagaggttcctgcaggattactttgatggcaatctgaagagatacctgaagtctgaacctatcccagagagcaatgatgggcctgtgaaggtagtggtagcagagaattttgatgaaatagtgaataatgaaaataaagatgtgctgattgaattttatgccccttggtgtggtcactgtaagaacctggagcccaagtataaagaacttggcgagaagctcagcaaagacccaaatatcgtcatagccaagatggatgccacagccaatgatgtgccttctccatatgaagtcagaggttttcctaccatatacttctctccagccaacaagaagctaaatccaaagaaatatgaaggtggccgtgaattaagtgattttattagctatctacaaagagaagctacaaacccccctgtaattcaagaagaaaaacccaagaagaagaagaaggcacaggaggatctctaaagcagtagccaaacaccactttgtaaaaggactcttccatcagagatgggaaaaccattggggaggactaggacccatatgggaattattacctctcagggccgagaggacagaatggatataatctgaatcctgttaaattttctctaaactgtttcttagctgcactgtttatggaaataccaggaccagtttatgtttgtggttttgggaaaaattatttgtgttgggggaaatgttgtgggggtggggttgagttgggggtattttctaattttttttgtacatttggaacagtgacaataaatgagacccctttaaactgtcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - protein, 505 aa, Homo sapiens
MRLRRLALFPGVALLLAAARLAAASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAQEDL

SEQ ID NO: 7 - DNA, 1899 bp, Homo sapiens
  CDS (69)...(1319)
gtccgtactgcagagccgctgccggagggtcgttttaaagggccgcgttgccgccccctcggcccgccatgctgctatccgtgccgctgctgctcggcctcctcggcctggccgtcgccgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagcttgccgcgccaaataatgtctctgtgagactcgagaactttcatttttttccaggctggttcggatttggggtggattttggttttgttcccctcctccactctcccccaccccctccccgccctttttttttttttttttaaactggtattttatcctttgattctccttcagccctcacccctggttctcatctttcttgatcaacatcttttcttgcctctgtgccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctc

SEQ ID NO: 8 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 9 - DNA, 1288 bp, Homo sapiens
  CDS (64)...(1215)
agaggcgcagagagagctgggagctaaggggtggcggcgaccggaagcgcagtgcacacccccatggcccgggctttggtccagttctgggccatatgcatgctgcgagtggcgctggctaccgtctatttccaagaggaatttctagacggagagcattggagaaaccgatggttgcagtccaccaatgactcccgatttgggcattttagactttcgtcgggcaagttttatggtcataaagagaaagataaaggtctgcaaaccactcagaatggccgattctatgccatctctgcacgcttcaaaccgttcagcaataaagggaaaactctggttattcagtacacagtaaaacatgagcagaagatggactgtggagggggctacattaaggtctttcctgcagacattgaccagaagaacctgaatggaaaatcgcaatactatattatgtttggacccgatatttgtggatttgatatcaagaaagttcatgttattttacatttcaagaataagtatcacgaaaacaagaaactgatcaggtgtaaggttgatggcttcacacacctgtacactctaattttaagaccagatctttcttatgatgtgaaaattgatggtcagtcaattgaatccggcagcatagagtacgactggaacttaacatcactcaagaaggaaacgtccccggcagaatcgaaggattgggaacagactaaagacaacaaagcccaggactgggagaagcattttctggacgccagcaccagcaagcagagcgactggaacggtgacctggatggggactggccagcgccgatgctccagaagcccccgtaccaggatggcctgaaaccagaaggtattcataaagacgtctggctccaccgtaagatgaagaataccgactatttgacgcagtatgacctctcagaatttgagaacattggtgccattggcctggagctttggcaggtgagatctggaaccatttttgataactttctgatcacagatgatgaagagtatgcagataattttggcaaggccacctggggcgaaaccaagggtccagaaagggagatggatgccatacaggccaaggaggaaatgaagaaggcccgcgaggaagaggaggaagagctgctgtcgggaaaaattaacaggcacgaacattacttcaatcaatttcacagaaggaatgaactttagtgatccccattggatataaggatgactggtaaaatctcattgctactttaatctaaaaaaaaaaaaaaaa

SEQ ID NO: 10 - protein, 384 aa, Homo sapiens
MARALVQFWAICMLRVALATVYFQEEFLDGEHWRNRWLQSTNDSRFGHFRLSSGKFYGHKEKDKGLQTTQNGRFYAISARFKPFSNKGKTLVIQYTVKHEQKMDCGGGYIKVFPADIDQKNLNGKSQYYIMFGPDICGFDIKKVHVILHFKNKYHENKKLIRCKVDGFTHLYTLILRPDLSYDVKIDGQSIESGSIEYDWNLTSLKKETSPAESKDWEQTKDNKAQDWEKHFLDASTSKQSDWNGDLDGDWPAPMLQKPPYQDGLKPEGIHKDVWLHRKMKNTDYLTQYDLSEFENIGAIGLELWQVRSGTIFDNFLITDDEEYADNFGKATWGETKGPEREMDAIQAKEEMKKAREEEEEELLSGKINRHEHYFNQFHRRNEL

SEQ ID NO: 11 - DNA, 1659 bp, Homo sapiens
  CDS (28)...(1560)
agcagtacaggcagaagctggcggctcatggcttcgtgcccatggggtcaggaacagggagcgaggagcccctcggaggagcctccagaggaggaaatccccaaggaggatgggatcttggtgctgagccgccacaccctgggcctggccctgcgggagcaccctgccctgctggtggaattctatgccccgtggtgtgggcactgccaggccctggcccccgagtacagcaaggcagctgccgtgctcgcggccgagtcaatggtggtcacgctggccaaggtggatgggcccgcgcagcgcgagctggctgaggagtttggtgtgacggagtaccctacgctcaagttcttccgcaatgggaaccgcacgcaccccgaggagtacacaggaccacgggacgctgagggcattgccgagtggctgcgacggcgggtggggcccagtgccatgcggctggaggatgaggcggccgcccaggcgctgatcggtggccgggacctagtggtcattggcttcttccaggacctgcaggacgaggacgtggccaccttcttggccttggcccaggacgccctggacatgacctttggcctcacagaccggccgcggctctttcagcagtttggcctcaccaaggacactgtggttctcttcaagaagtttgatgaggggcgggcagacttccccgtggacgaggagcttggcctggacctgggggatctgtcgcgcttcctggtcacacacagcatgcgcctggtcacggagttcaacagccagacgtctgccaagatcttcgcggccaggatcctcaaccacctgctgctgtttgtcaaccagacgctggctgcgcaccgggagctcctagcgggctttggggaggcagctccccgcttccgggggcaggtgctgttcgtggtggtggacgtggcggccgacaatgagcacgtgctgcagtactttggactcaaggctgaggcagcccccactctgcgcttggtcaaccttgaaaccactaagaagtatgcgcctgtggatgggggccctgtcaccgcagcgtccatcactgctttctgccatgcagtcctcaacggccaagtcaagccctatctcctgagccaggagataccccctgattgggatcagcggccagttaagaccctcgtgggcaagaattttgagcaggtggcttttgacgaaaccaagaatgtgtttgtcaagttctatgccccgtggtgcacccactgcaaggagatggcccctgcctgggaggcattggctgagaagtaccaagaccacgaggacatcatcattgctgagctggatgccacggccaacgagctggatgccttcgctgtgcacggcttccctactctcaagtacttcccagcagggccaggtcggaaggtgattgaatacaaaagcaccagggacctggagactttctccaagttcctggacaacgggggcgtgctgcccacggaggagtccccggaggagccagcagccccgttcccggagccaccggccaactccactatggggtccaaggaggaactgtagctgcccccgtgtcacccccgccatcactgctggacaggagccacccccttgggtaccagagggagctgtgcattgtgaataaagagtgagcttggt

SEQ ID NO: 12 - protein, 511 aa, Homo sapiens
MASCPWGQEQGARSPSEEPPEEEIPKEDGILVLSRHTLGLALREHPALLVEFYAPWCGHCQALAPEYSKAAAVLAAESMVVTLAKVDGPAQRELAEEFGVTEYPTLKFFRNGNRTHPEEYTGPRDAEGIAEWLRRRVGPSAMRLEDEAAAQALIGGRDLVVIGFFQDLQDEDVATFLALAQDALDMTFGLTDRPRLFQQFGLTKDTVVLFKKFDEGRADFPVDEELGLDLGDLSRFLVTHSMRLVTEFNSQTSAKIFAARILNHLLLFVNQTLAAHRELLAGFGEAAPRFRGQVLFVVVDVAADNEHVLQYFGLKAEAAPTLRLVNLETTKKYAPVDGGPVTAASITAFCHAVLNGQVKPYLLSQEIPPDWDQRPVKTLVGKNFEQVAFDETKNVFVKFYAPWCTHCKEMAPAWEALAEKYQDHEDIIIAELDATANELDAFAVHGFPTLKYFPAGPGRKVIEYKSTRDLETFSKFLDNGGVLPTEESPEEPAAPFPEPPANSTMGSKEEL

SEQ ID NO: 13 - DNA, 2865 bp, Homo sapiens
  CDS (46)...(1980)
ccagcggccgccgacgctaggaggccgcgctccgcccccgctaccatgaggccccggaaagccttcctgctcctgctgctcttggggctggtgcagctgctggccgtggcgggtgccgagggcccggacgaggattcttctaacagagaaaatgccattgaggatgaagaggaggaggaggaggaagatgatgatgaggaagaagacgacttggaagttaaggaagaaaatggagtcttggtcctaaatgatgcaaactttgataattttgtggctgacaaagacacagtgctgctggagttttatgctccatggtgtggacattgcaagcagtttgctccggaatatgaaaaaattgccaacatattaaaggataaagatcctcccattcctgttgccaagatcgatgcaacctcagcgtctgtgctggccagcaggtttgatgtgagtggctaccccaccatcaagatccttaagaaggggcaggctgtagactacgagggctccagaacccaggaagaaattgttgccaaggtcagagaagtctcccagcccgactggacgcctccaccagaagtcacgcttgtgttgaccaaagagaactttgatgaagttgtgaatgatgcagatatcattctggtggagttttatgccccatggtgtggacactgcaagaaacttgcccccgagtatgagaaggccgccaaggagctcagcaagcgttctcctccaattcccctggcaaaggtcgacgccaccgcagaaacagacctggccaagaggtttgatgtctctggctatcccaccctgaaaattttccgcaaaggaaggccttatgactacaacggcccacgagaaaaatatggaatcgttgattacatgatcgagcagtccgggcctccctccaaggagattctgaccctgaagcaggtccaggagttcctgaaggatggagacgatgtcatcatcatcggggtctttaagggggagagtgacccagcctaccagcaataccaggatgccgctaacaacctgagagaagattacaaatttcaccacactttcagcacagaaatagcaaagttcttgaaagtctcccaggggcagttggttgtaatgcagcctgagaaattccagtccaagtatgagccccggagccacatgatggacgtccagggctccacccaggactcggccatcaaggacttcgtgctgaagtacgccctgcccctggttggccaccgcaaggtgtcaaacgatgctaagcgctacaccaggcgccccctggtggtcgtctactacagtgtggacttcagctttgattacagagctgcaactcagttttggcggagcaaagtcctagaggtggccaaggacttccctgagtacacctttgccattgcggacgaagaggactatgctggggaggtgaaggacctggggctcagcgagagtggggaggatgtcaatgccgccatcctggacgagagtgggaagaagttcgccatggagccagaggagtttgactctgacaccctccgcgagtttgtcactgctttcaaaaaaggaaaactgaagccagtcatcaaatcccagccagtgcccaagaacaacaagggacccgtcaaggtcgtggtgggaaagacctttgactccattgtgatggaccccaagaaggacgtcctcatcgagttctacgcgccatggtgcgggcactgcaagcagctagagcccgtgtacaacagcctggccaagaagtacaagggccaaaagggcctggtcatcgccaagatggacgccactgccaacgacgtccccagcgaccgctataaggtggagggcttccccaccatctacttcgcccccagtggggacaaaaagaacccagttaaatttgagggtggagacagagatctggagcatttgagcaagtttatagaagaacatgccacaaaactgagcaggaccaaggaagagctttgaaggcctgaggtctgcggaaggtgggaggaggcagacgccctgcgtggcccatggtcggggcgtccaccggaggccggcaacaaacgacagtatctcggattcctttttttttttttttaattttttatactttgttgtttcacttcatgctctgaatactgaataaccatgaatgactgaatagtttagtccagatttttacagaggatacatctatttttatcattatttggggtttgaaaaatttttttttacaccttctaatttctttatttctcaaagcagataattcttctgtgtgaaaatgttttctttttttaatttaaggtttaaaattccttttccaaatcatgttgattttgctctttaaaaaaaaaaaaaaaaaaaaaaaaaaaaaaaaaaaaagaagggctgggaccaaccgggtgagatccacaagtctctggatgtggctgaaggcaaatacacaattgaagtactttctgttttgaagtgctttcccttttgaatctggtttgaaacatgcagcttctgtctctagcccaaggaaagaccaaaacatagggaaataaaagcatttatctttgtcttggaagtaattgttgaagttgtgcagttgatcagtgcacagttagctgcaatgtttatagaaattgattgttaaaccaaatttacactggcatgtgtggtgtagtttctaaaaggcacttcacatttgaaatttttcttaccttagaaagtttctagtgatctaaatgtctagttttgtattcttttgtgtgtgttcactgtttctcagtattaccacttgaataattctctgtacaggggggtttgtgctatacactgggatgtctaattgcagcaataaagcctttcttt

SEQ ID NO: 14 - protein, 645 aa, Homo sapiens
MRPRKAFLLLLLLGLVQLLAVAGAEGPDEDSSNRENAIEDEEEEEEEDDDEEEDDLEVKEENGVLVLNDANFDNFVADKDTVLLEFYAPWCGHCKQFAPEYEKIANILKDKDPPIPVAKIDATSASVLASRFDVSGYPTIKILKKGQAVDYEGSRTQEEIVAKVREVSQPDWTPPPEVTLVLTKENFDEVVNDADIILVEFYAPWCGHCKKLAPEYEKAAKELSKRSPPIPLAKVDATAETDLAKRFDVSGYPTLKIFRKGRPYDYNGPREKYGIVDYMIEQSGPPSKEILTLKQVQEFLKDGDDVIIIGVFKGESDPAYQQYQDAANNLREDYKFHHTFSTEIAKFLKVSQGQLVVMQPEKFQSKYEPRSHMMDVQGSTQDSAIKDFVLKYALPLVGHRKVSNDAKRYTRRPLVVVYYSVDFSFDYRAATQFWRSKVLEVAKDFPEYTFAIADEEDYAGEVKDLGLSESGEDVNAAILDESGKKFAMEPEEFDSDTLREFVTAFKKGKLKPVIKSQPVPKNNKGPVKVVVGKTFDSIVMDPKKDVLIEFYAPWCGHCKQLEPVYNSLAKKYKGQKGLVIAKMDATANDVPSDRYKVEGFPTIYFAPSGDKKNPVKFEGGDRDLEHLSKFIEEHATKLSRTKEEL

SEQ ID NO: 15 - DNA, 2412 bp, Homo sapiens
  CDS (1)...(2409)
atgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcctgaagaagaacctgaagagacagcagaagacacaacagaagacacagagcaagacgaagatgaagaaatggatgtgggaacagatgaagaagaagaaacagcaaaggaatctacagctgaaaaagatgaattgtaa

SEQ ID NO: 16 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 17 - DNA, 21 bp, Homo sapiens
aacaactgcatgggtaacctt